(19) United States
(12) Patent Application Publication
(10) Pub. No.: US 2009/0093485 A1
(43) Pub. Date: Apr. 9, 2009

(54) Novel Sulphonamide Derivatives as Glucocorticoid Receptor Modulators for the Treatment of Inflammatory Diseases

(75) Inventors: Hakan Bladh, Lund (SE); Krister Henriksson, Lund (SE); Vijaykumar Hulikal, Bangalore (IN); Matti Lepisto, Lund (SE)

Correspondence Address: FISH & RICHARDSON P.C., P.O BOX 1022, MINNEAPOLIS, MN, 55440-1022, US

(73) Assignee: ASTRAZENECA AB, Södertälje (SE)

(21) Appl. No.: 11/718,214

(22) PCT Filed: Oct. 26, 2005
(86) PCT No.: PCT/SE2005/001610

§ 371 Date: Feb. 15, 2008

(30) Foreign Application Priority Data
Oct. 29, 2004 (SE) 0402636-5
Mar. 22, 2005 (SE) 0500651-5

(51) Int. Cl. A61K 31/53; C07D 215/38; A61K 31/47; C07D 307/78; A61K 31/343; C07D 209/04; A61K 31/404; C07C 311/15; A61K 31/18; C07D 277/20; A61K 31/426; C07D 239/24; A61K 31/505; C07D 213/02; A61K 31/44; C07D 405/02; A61K 31/443; C07D 251/02; C07D 239/72; A61K 31/517; A61P 29/00

(52) U.S. Cl. 514/245; 546/160; 514/313; 549/467; 514/469; 548/504; 514/415; 564/84; 514/602; 548/195; 514/371; 544/297; 514/272; 546/329; 514/357; 546/284.1; 514/337; 544/204; 544/287; 514/266.3

(57) ABSTRACT

Compounds of formula (I) or a pharmaceutically acceptable salt thereof; compositions comprising them, processes for preparing them and their use in medical therapy (for example modulating the glucocorticoid receptor in a warm blooded animal).

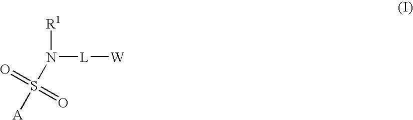

The present invention relates to sulphonamide derivatives, to their use as medicaments (for example in the treatment of an inflammatory disease state), to pharmaceutical compositions comprising them and to processes for preparing them.

Sulphonamide derivatives are disclosed as anti-inflammatories in WO 2004/019935 and WO 2004/050631. Pharmaceutically active sulphonamides are also disclosed in Arch. Pharm. (1980) 313 166-173, J. Med. Chem. (2003) 46 64-73, J. Med. Chem. (1997) 40 996-1004, EP 0031954, EP 1190710 (WO 200124786), U.S. Pat. No. 5,861,401, U.S. Pat. No. 4,948,809, U.S. Pat. No. 3,992,441 and WO 99/33786.

It is known that certain non-steroidal compounds interact with the glucocorticoid receptor (GR) and, as a result of this interaction, produce a suppression of inflammation (see, for example, U.S. Pat. No. 6,323,199). Such compounds can show a clear dissociation between anti-inflammatory and metabolic actions making them superior to earlier reported steroidal and non-steroidal glucocorticoids. The present invention provides further non-steroidal compounds as modulators (for example agonists, antagonists, partial agonists or partial antagonists) of the glucocorticoid receptor capable of having a dissociation between their anti-inflammatory and metabolic actions.

The present invention provides a compound of formula (I):

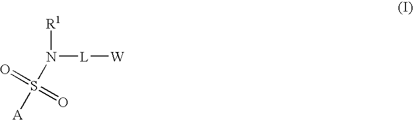

wherein:
A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(CM alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl(optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹);
R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl;
R¹ is hydrogen, C₁₋₆ alkyl, phenyl, pyridinylC(O), C₃₋₆ cycloalkyl, (C₃₋₆ cycloalkyl)CH₂ or C₃₋₄ alkenyl;
L is a bond, C₁₋₄ alkylene (optionally substituted by C₁₋₄ alkyl or C₁₋₄ haloalkyl), C₁₋₄ alkylene-NH (optionally substituted by C₁₋₄ alkyl or C₁₋₄ haloalkyl), CH₂C(O)NH, CH(CH₃)C(O)NH, C₁₋₄ alkylene-O (optionally substituted by C₁₋₄ alkyl or C₁₋₄ haloalkyl), C₁₋₄ alkylene-S (optionally substituted by C₁₋₄ alkyl or C₁₋₄ haloalkyl), C₁₋₄ alkylene-S(O) (optionally substituted by C₁₋₄ alkyl or C₁₋₄ haloalkyl) or C₁₋₄ alkylene-S(O)₂ (optionally substituted by C₁₋₄ alkyl or C₁₋₄ haloalkyl);
W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl;
W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ allyl), C(O)N(C₁₋₄ alkyl)₂, NHC(O)(C₁₋₄ alkyl) or NR¹²R¹³;
R¹² and R¹³ are, independently, hydrogen, C₁₋₄ alkyl or C₃₋₇ cycloalkyl;
or a pharmaceutically acceptable salt thereof.

Compounds of formula (I) can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate, p-toluenesulphonate, succinate, glutarate or malonate.

The compounds of formula (I) may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl.

Haloalkyl comprises, for example, 1 to 6, such as 1, 2, 3, 4 or 5 halogen (such as fluorine or chlorine) atoms. It is, for example, CHF₂, CF₃, CH₂CF₃, C₂F₅ or CH₂Cl. Haloalkoxy comprises, for example, 1 to 6, such as 1, 2, 3, 4 or 5 halogen (such as fluorine or chlorine) atoms. It is, for example, OCHF₂, OCF₃, OCH₂CF₃, OC₂F₅ or OCH₂Cl.

Fluoroalkyl comprises, for example, 1 to 6, such as 1, 2, 3, 4 or 5 fluorine atoms. It is, for example, CHF₂, CF₃, CH₂CF₃ or C₂F₅. Fluoroalkoxy comprises, for example, 1 to 6, such as 1, 2, 3, 4 or 5 fluorine atoms. It is, for example, OCHF₂, OCF₃, OCH₂CF₃ or OC₂F₅.

Cycloalkyl is for example, cyclopropyl, cyclopentyl or cyclohexyl.

In one particular aspect the present invention provides a compound of formula (I), wherein A is phenyl, naphthyl, pyridinyl, thienyl, quinolinyl or isoquinolinyl, and A is optionally substituted by halo, C₁₋₆ alkyl, C₁₋₆ alkoxy, C₁₋₄ alkylthio, CF₃, OCF₃, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, CF₃, OCF₃, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ allyl), benzyloxy, C(O)NH₂, C(O)NH(C₁₋₄ allyl), C(O)N(C₁₋₄ alkyl)₂, NHC(O)(C₁₋₄ alkyl) or NR¹⁴R¹⁵) or phenyl (optionally substituted by halo, C₁₋₆ alkyl, C₁₋₆ alkoxy, C₁₋₄ alkylthio, CF₃, OCF₃, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷); R¹⁰, R¹¹, R¹⁴, R¹⁵, R¹⁶ and R¹⁷ are, independently, hydrogen, C₁₋₄ alkyl or C₃₋₇ cycloalkyl; R¹ is hydrogen, C₁₋₆ alkyl, phenyl, pyridylC(O), C₃₋₆ cycloalkyl, (C₃₋₆ cycloalkyl)CH₂ or C₃₋₄ alkenyl; L is a bond, C₁₋₄ alkylene (optionally substituted by C₁₋₄ alkyl), C₁₋₄ alkylene-NH (optionally substituted by C₁₋₄ alkyl), CH₂C(O)NH, CH(CH₃)C(O)NH, C₁₋₄ alkylene-O (optionally substituted by C₁₋₄ alkyl); C₁₋₄ alkylene-S (optionally substituted by C₁₋₄ alkyl); C₁₋₄ alkylene-S(O) (optionally substituted by C₁₋₄ alkyl); C₁₋₄ alkylene-S(O)₂ (optionally substituted by C₁₋₄ alkyl); W is phenyl, methylenedioxyphenyl, thiazolyl, isoxazolyl, pyridyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl or [1,6]-naphthiridinyl; W is optionally substituted by halo, C₁₋₄ alkyl, C₁₋₆ alkoxy, C₁₋₄ alkylthio, CF₃) OCF₃, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof; for use as a medicament.

In another aspect the present invention provides a compound of formula (I), wherein A is phenyl, naphthyl, thienyl, quinolinyl or isoquinolinyl, and A is optionally substituted by halo, C₁₋₆ alkyl, C₁₋₆ alkoxy, C₁₋₄ alkylthio, CF₃, OCF₃, phenoxy (optionally substituted by halo or C₁₋₄ alkyl), phenyl (optionally substituted by halo or C₁₋₄ alkyl), pyridinyloxy, benzyloxy, nitro, cyano, S(O)₂NH₂, C(O)(C₁₋₄ alkyl), C(O)NH₂, NHC(O)(C₁₋₄ alkyl) or NR¹⁰R¹¹; R¹⁰ and R¹¹ are, independently, hydrogen, C₁₋₄ alkyl or C₃₋₇ cycloalkyl; R¹ is hydrogen, C₁₋₆ alkyl, phenyl, pyridylC(O), cyclohexyl, cyclohexylCH₂ or C₃₋₄ alkenyl; L is a bond, C₁₋₄ alkylene (optionally substituted by C₁₋₄ alkyl), C₁₋₄ alkylene-NH (optionally substituted by C₁₋₄ alkyl), CH₂C(O)NH or C₁₋₄ alkylene-O (optionally substituted by C₁₋₄ alkyl); W is phenyl, benzofuranyl, indolyl, tetrahydroquinolinyl, thiazolyl, pyridyl, isoxazolyl, pyrimidinyl or 1,3,5-triazinyl, and W is optionally substituted by halo, C₁₋₆ alkyl, C₁₋₆ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof; for use as a medicament.

In a further aspect the present invention provides a compound of formula (I) wherein: A is phenyl, naphthyl, thienyl, quinolinyl or isoquinolinyl, and A is optionally substituted by halo (such as fluoro, chloro or bromo), C₁₋₆ alkyl, C₁₋₆ alkoxy, nitro, phenoxy (optionally substituted by C₁₋₄ alkyl), phenyl (optionally substituted by halo (such as fluoro)), pyridinyloxy or N(C₁₋₄ alkyl)₂; R¹ is hydrogen, C₁₋₆ alkyl, phenyl, pyridylC(O), cyclohexyl, cyclohexylCH₂ or C₃₋₄ alkenyl, L is a bond, C₁₋₄ alkylene (optionally substituted by C₁₋₄ alkyl), C₁₋₄ alkylene-NH (optionally substituted by C₁₋₄ alkyl), CH₂C(O)NH or C₁₋₄ alkylene-O (optionally substituted by C₁₋₄ alkyl); W is phenyl, benzofuranyl, indolyl, tetrahydroquinolinyl, thiazolyl, pyridyl, isoxazolyl, pyrimidinyl or 1,3,5-triazinyl, and W is optionally substituted by halo (such as chloro or bromo), C₁₋₆ alkyl, C₁₋₆ alkoxy, C(O)(C₁₋₄ alkyl), S(O)₂NH₂, NO₂, CO₂(C₁₋₄ alkyl) or N(C₁₋₄ alkyl)₂; or a pharmaceutical acceptable salt thereof; for use as a medicament.

In another aspect the present invention provides a compound of formula (I) wherein A is phenyl, naphthyl, pyridinyl, thienyl, quinolinyl or isoquinolinyl, and A is optionally substituted by halo, C₁₋₆ alkyl, C₁₋₆ alkoxy, C₁₋₄ alkylthio, CF₃, OCF₃, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, CF₃, OCF₃, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵) or phenyl (optionally substituted by halo, C₁₋₆ allyl, C₁₋₆ alkoxy, C₁₋₄ alkylthio, CF₃, OCF₃, nitro, cyano, C(O)₂H, C(O)₂(C₁₋₄ allyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷); R¹⁰, R¹¹, R¹⁴, R¹⁵, R¹⁶ and R¹⁷ are, independently, hydrogen, C₁₋₄ alkyl or C₃₋₇ cycloalkyl; R¹ is hydrogen, C₁₋₆ alkyl, phenyl, pyridylC(O), C₃₋₆ cycloalkyl, (C₃₋₆ cycloalkyl)CH₂ or C₃₋₄ alkenyl; L is a bond, C₁₋₄ alkylene (optionally substituted by C₁₋₄ alkyl), C₁₋₄ alkylene-NH (optionally substituted by C₁₋₄ alkyl), CH₂C(O)NH, CH(CH₃)C(O)NH, C₁₋₄ alkylene-O (optionally substituted by C₁₋₄ alkyl); C₁₋₄ alkylene-S (optionally substituted by C₁₋₄ alkyl); C₁₋₄ alkylene-S(O) (optionally substituted by C₁₋₄ alkyl); C₁₋₄ alkylene-S(O)₂ (optionally substituted by C₁₋₄ alkyl); W is phenyl, methylenedioxyphenyl, thiazolyl, isoxazolyl, pyridyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl or [1,6]-naphthiridinyl; W is optionally substituted by halo, C₁₋₆ alkyl, C₁₋₆ alkoxy, C₁₋₄ alkylthio, CF₃, OCF₃, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof.

In a further aspect the present invention provides a compound of formula (I) wherein: A is phenyl, naphthyl, thienyl, quinolinyl or isoquinolinyl, and A is optionally substituted by halo, C₁₋₆ alkyl, C₁₋₆ alkoxy, C₁₋₄ alkylthio, CF₃, OCF₃, phenoxy (optionally substituted by halo or C₁₋₄ alkyl), phenyl (optionally substituted by halo or C₁₋₄ alkyl), pyridinyloxy, benzyloxy, nitro, cyano, S(O)₂NH₂, C(O)(C₁₋₄ alkyl), C(O)NH₂, NHC(O)(C₁₋₄ alkyl) or NR¹⁰R¹¹; R¹⁰ and R¹¹ are, independently, hydrogen, C₁₋₄ alkyl or C₃₋₇ cycloalkyl; R¹ is hydrogen, C₁₋₆ alkyl, phenyl, pyridylC(O), cyclohexyl, cyclohexylCH₂ or C₃₋₄ alkenyl; L is a bond, C₁₋₄ alkylene (optionally substituted by C₁₋₄ alkyl), C₁₋₄ alkylene-NH (optionally substituted by C₁₋₄ alkyl), CH₂C(O)NH or C₁₋₄ alkylene-O (optionally substituted by C₁₋₄ alkyl); W is phenyl, benzofuranyl, indolyl, tetrahydroquinolinyl, thiazolyl, pyridyl, isoxazolyl, pyrimidinyl or 1,3,5-triazinyl, and W is optionally substituted by halo, C₁₋₆ alkyl, C₁₋₆ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof.

In a still further aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)).

In another aspect the invention provides a compound of formula (I) wherein L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl), CH₂C(O)NH, CH(CH₃)C(O)NH, C₂₋₄ alkylene-O (substituted by C₁₋₄ alkyl or C₁₋₄ haloalkyl), C₂₋₄ alkylene-S (substituted by C₁₋₄ alkyl or C₁₋₄ haloalkyl), C₂₋₄ alkylene-S(O) (optionally substituted by C₁₋₄ alkyl or C₁₋₄ haloalkyl) or C₂₋₄ alkylene-S(O)₂ (optionally substituted by C₁₋₄ alkyl or C₁₋₄ haloalkyl); wherein C₁₋₄ alkyl is, for example, methyl or ethyl; and C₁₋₄ haloalkyl is, for example, CF₃.

In yet another aspect the invention provides a compound of formula (I) wherein L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl) or C₂₋₄ alkylene-O (substituted by C₁₋₄ alkyl or C₁₋₄ haloalkyl); wherein C₁₋₄ alkyl is, for example, methyl or ethyl; and C₁₋₄ haloalkyl is, for example, CF₃.

In a further aspect the invention provides a compound of formula (I) wherein L is C₃ alkylene (substituted by C₁₋₄ alkyl), C₂ alkylene-NH (substituted by C₁₋₄ alkyl) or C₂ alkylene-O (substituted by C₁₋₄ alkyl); wherein C₁₋₄ alkyl is, for example, methyl or ethyl. L is, for example, C₂ alkylene-NH (substituted by C₁₋₄ alkyl). L is, for example, C₂ alkylene-O (substituted by C₁₋₄ alkyl).

In a still further aspect the invention provides a compound of formula (I) wherein L is CH(CH₃)CH₂CH₂ (such as in the S-configuration), CH(CH₃)CH₂NH (such as in the S-configuration), CH(CH₃)CH₂O (such as in the S-configuration), CH(C₂H₅)CH₂CH₂ (such as in the S-configuration), CH(C₂H₅)CH₂NH (such as in the S-configuration), CH(C₂H₅)CH₂O (such as in the S-configuration) or CH(CF₃)CH₂CH₂ (such as in the S-configuration).

In another aspect the present invention provides a compound of formula (I) wherein L is CH(CH₃)CH₂NH (such as in the S-configuration) or it provides a compound of formula (I) wherein L is CH(CH₃)CH₂O (such as in the S-configuration).

In yet another aspect the present invention provides a compound of formula (I) wherein W is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl).

In a further aspect the present invention provides a compound of formula (I) wherein W is indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl).

In a still further aspect the present invention provides a compound of formula (I) wherein W is indol-4-yl, indol-5-yl, indol-6-yl, indol-7-yl, indazol-4-yl, indazol-5-yl, indazol-6-yl, indazol-7-yl, quinolin-5-yl or isoquinolin-5-yl.

In another aspect the present invention provides a compound of formula (I) wherein W is indazol-4-yl, indazol-5-yl, indazol-6-yl, indazol-7-yl or quinolin-5-yl.

In yet another aspect the present invention provides a compound of formula (I) wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In a further aspect the present invention provides a compound of formula (I) wherein L is C₁₋₄ alkylene (optionally substituted by C₁₋₄ alkyl) or C₁₋₄ alkylene-O (optionally substituted by C₁₋₄ alkyl); for example L is CH(CH₃)CH₂O, CH₂CH₂O, CH(CH₃)(CH₂)₂ or (CH₂)₃.

In another aspect of the invention L is C₁₋₄ alkylene (optionally substituted by C₁₋₄ alkyl) or C₁₋₄ alkylene-O (optionally substituted by C₁₋₄ alkyl).

In yet another aspect the present invention provides a compound of formula (I) wherein R¹ is hydrogen.

In a still further aspect the present invention provides a compound of formula (I) wherein W is methylenedioxyphenyl, benzofuranyl, benzthienyl, indolyl, indolinyl, dihydroindolinyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl or [1,6]-naphthiridinyl, optionally substituted as defined above. In another aspect of the invention W is linked to L by a ring-carbon in the benzene ring part of its structure (see for example, Example 77, 78, 79, 80 or 83).

In a still further aspect the present invention provides a compound of formula (I) wherein: A is phenyl, naphthyl or thienyl, and A is optionally substituted by halo, C₁₋₆ alkyl, C₁₋₆ alkoxy, C₁₋₄ alkylthio, CF₃, OCF₃, phenoxy (optionally substituted by halo or C₁₋₄ alkyl), phenyl (optionally substituted by halo or C₁₋₄ alkyl), pyridinyloxy, benzyloxy, nitro, cyano, S(O)₂NH₂, C(O)(C₁₋₄ alkyl), C(O)NH₂, NHC(O)(C₁₋₄ alkyl) or NR¹⁰R¹¹; R¹⁰ and R¹¹ are, independently, hydrogen, C₁₋₄ alkyl or C₃₋₇ cycloalkyl; R¹ is hydrogen; L is C₁₋₄ alkylene (optionally substituted by C₁₋₄ alkyl) or C₁₋₄ alkylene-O (optionally substituted by C₁₋₄ alkyl); W is phenyl optionally substituted by halo, C₁₋₆ alkyl, C₁₋₆ alkoxy, C₁₋₄ alkylthio, CF₃, OCF₃, benzyloxy, nitro, cyano, S(O)₂NH₂, C(O)(C₁₋₄ alkyl), C(O)NH₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof.

In a still further aspect the present invention provides a compound of formula (I) wherein: A is phenyl, naphthyl or thienyl, and A is optionally substituted by halo, C₁₋₆ alkyl, C₁₋₆ alkoxy, CF₃, OCF₃, phenoxy (optionally substituted by halo or C₁₋₄ alkyl), phenyl (optionally substituted by halo or C₁₋₄ alkyl), pyridinyloxy, nitro or cyano; R¹ is hydrogen; L is C₁₋₄ alkylene (optionally substituted by C₁₋₄ alkyl) or C₁₋₄ alkylene-O (optionally substituted by C₁₋₄ alkyl); W is phenyl optionally substituted by halo, C₁₋₆ alkyl, C₁₋₆ alkoxy, CF₃, OCF₃, nitro or cyano; or a pharmaceutically acceptable salt thereof.

In another aspect the present invention provides a compound of formula (I) wherein A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ allyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ allyl), S(O)₂N(C₁₋₄ alkyl)₂, C(O)(C₁₋₄ alkyl), benzyloxy, C(O)NH₂, C(O)NH(C₁₋₄ alkyl), C(O)N(CM alkyl)₂, NHC(O)(C₁₋₄ alkyl) or NR¹⁸R¹⁹) or pyrazolyl(optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹); R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl; R¹ is hydrogen; L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl) or C₂₋₄ alkylene-O (substituted by C₁₋₄ alkyl or C₁₋₄ haloalkyl) {for example L is C₃ alkylene (substituted by C₁₋₄ alkyl), C₂ alkylene-NH (substituted by C₁₋₄ alkyl) or C₂ alkylene-O (substituted by C₁₋₄ alkyl)}; W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl; W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof {for example the compound is not in the form of a salt}.

In yet another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl) or C₂₋₄ alkylene-O (substituted by C₁₋₄ alkyl or C₁₋₄ haloalkyl) {for example L is C₃ alkylene (substituted by C₁₋₄ alkyl), C₂ alkylene-NH (substituted by C₁₋₄ alkyl) or C₂ alkylene-O (substituted by C₁₋₄ alkyl)}; W is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl) {for example W is indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl)}; wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In a further aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl) or C₂₋₄ alkylene-O (substituted by C₁₋₄ alkyl or C₁₋₄ haloalkyl) {for example L is C₃ alkylene (substituted by C₁₋₄ alkyl), C₂ alkylene-NH (substituted by C₁₋₄ alkyl) or C₂ alkylene-O (substituted by C₁₋₄ alkyl)}; W is indazol-4-yl, indazol-5-yl, indazol-6-yl, indazol-7-yl or quinolin-5-yl; wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃).

In another aspect the present invention provides a compound of formula (I) wherein A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ allyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ allyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ allyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹); R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl; R¹ is hydrogen; L is CH(CH₃)CH₂CH₂ (such as in the S-configuration), CH(CH₃)CH₂NH (such as in the S-configuration), CH(CH₃)CH₂O (such as in the S-configuration), CH(C₂H₅)CH₂CH₂ (such as in the S-configuration), CH(C₂H₅)CH₂NH (such as in the S-configuration), CH(C₂H₅)CH₂O (such as in the S-configuration) or CH(CF₃)CH₂CH₂ (such as in the S-configuration); W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl; W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof {for example the compound is not in the form of a salt}.

In yet another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is CH(CH₃)CH₂CH₂ (such as in the S-configuration), CH(CH₃)CH₂NH (such as in the S-configuration), CH(CH₃)CH₂O (such as in the S-configuration), CH(C₂H₅)CH₂CH₂ (such as in the S-configuration), CH(C₂H₅)CH₂NH (such as in the S-configuration), CH(C₂H₅)CH₂O (such as in the S-configuration) or CH(CF₃)CH₂CH₂ (such as in the S-configuration); W is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl) {for example W is indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl)}; wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

In a further aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is CH(CH₃)CH₂CH₂ (such as in the S-configuration), CH(CH₃)CH₂NH (such as in the S-configuration), CH(CH₃)CH₂O (such as in the S-configuration), CH(C₂H₅)CH₂CH₂ (such as in the S-configuration), CH(C₂H₅)CH₂NH (such as in the S-configuration), CH(C₂H₅)CH₂O (such as in the S-configuration) or CH(CF₃)CH₂CH₂ (such as in the S-configuration); W is indazol-4-yl, indazol-5-yl, indazol-6-yl, indazol-7-yl or quinolin-5-yl; wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃).

In another aspect the present invention provides a compound of formula (I) wherein A is phenyl, naphthyl, pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl(optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR²⁰R²¹); R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl; R¹ is hydrogen; L is CH(CH₃)CH₂NH (such as in the S-configuration) or L is CH(CH₃)CH₂O (such as in the S-configuration); W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]-naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl; W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl, C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³; R¹² and R¹³ are, independently, hydrogen, C₁₋₄ alkyl or C₃₋₇ cycloalkyl; or a pharmaceutically acceptable salt thereof {for example the compound is not in the form of a salt}.

In yet another aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is CH(CH₃)CH₂NH (such as in the S-configuration) or L is CH(CH₃)CH₂O (such as in the S-configuration); W is phenyl, pyridyl, indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl) {for example W is indolyl (for example indol-4-yl, indol-5-yl, indol-6-yl or indol-7-yl), indazolyl (for example indazol-4-yl, indazol-5-yl, indazol-6-yl or indazol-7-yl), quinolinyl (for example quinolin-5-yl) or isoquinolinyl (for example isoquinolin-5-yl)}; wherein W is optionally substituted by halogen, C₁₋₄ alkyl, CF₃, C₁₋₄ alkoxy, OCF₃, phenyl (itself optionally substituted by halogen, CM alkyl, CF₃, C₁₋₄ alkoxy or OCF₃) or C(O)NH₂.

In a further aspect the present invention provides a compound of formula (I) wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)); R¹ is hydrogen; L is CH(CH₃)CH₂NH (such as in the S-configuration) or L is CH(CH₃)CH₂O (such as in the S-configuration); W is indazol-4-yl, indazol-5-yl, indazol-6-yl, indazol-7-yl or quinolin-5-yl; wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃).

In a still further aspect the present invention provides a compound:
4-Bromo-N-(1-methyl-3-phenyl-propyl)-benzenesulfonamide;
4-Chloro-N-(1-methyl-3-phenyl-propyl)-benzenesulfonamide;
4-Bromo-2-methyl-N-(1-methyl-3-phenyl-propyl)-benzenesulfonamide;
N-(1-Methyl-3-phenyl-propyl)-4-trifluoromethoxy-benzenesulfonamide;
4-Methoxy-2,3,6-trimethyl-N-(1-methyl-3-phenyl-propyl)-benzenesulfonamide;
4-tert-Butyl-N-(1-methyl-3-phenyl-propyl)-benzenesulfonamide;
N-(1-Methyl-3-phenyl-propyl)-4-phenoxy-benzenesulfonamide;
4′-Fluoro-biphenyl-4-sulfonic acid (1-methyl-3-phenyl-propyl)-amide;
N-(1-Methyl-3-phenyl-propyl)-4-propyl-benzenesulfonamide;
N-(1-Methyl-3-phenyl-propyl)-4-trifluoromethyl-benzenesulfonamide;
4-(1,1-Dimethyl-propyl)-N-(1-methyl-3-phenyl-propyl)-benzenesulfonamide;
N-(1-Methyl-3-phenyl-propyl)-3-trifluoromethyl-benzenesulfonamide;
Biphenyl-4-sulfonic acid (1-methyl-3-phenyl-propyl)-amide;
5-Bromo-thiophene-2-sulfonic acid (1-methyl-3-phenyl-propyl)-amide;
4-n-Butoxy-N-(1-methyl-3-phenyl-propyl)-benzenesulfonamide;
2,4,6-Trimethyl-N-(1-methyl-3-phenyl-propyl)-benzenesulfonamide;
N-(1-Methyl-3-phenyl-propyl)-3-p-tolyloxy-benzenesulfonamide;
N-[2-(2,6-Dimethyl-phenoxy)-1-methyl-ethyl]-3-nitro-benzenesulfonamide;
4-Bromo-N-[2-(2,6-dimethyl-phenoxy)-1-methyl-ethyl]-benzenesulfonamide;
N-{4-[2-(2,6-Dimethyl-phenoxy)-1-methyl-ethylsulfamoyl]-phenyl}-acetamide;
N-[2-(2,6-Dimethyl-phenoxy)-1-methyl-ethyl]-4-nitro-benzenesulfonamide;
4-Bromo-N-[2-(2,6-dimethyl-phenoxy)-1-methyl-ethyl]-2-methyl-benzenesulfonamide;
N-[2-(2,6-Dimethyl-phenoxy)-1-methyl-ethyl]-4-methoxy-benzenesulfonamide;
N-[2-(2,6-Dimethyl-phenoxy)-1-methyl-ethyl]-4-trifluoromethoxy-benzenesulfonamide;
4-tert-Butyl-N-[2-(2,6-dimethyl-phenoxy)-1-methyl-ethyl]-benzenesulfonamide;
4-Cyano-N-[2-(2,6-dimethyl-phenoxy)-1-methyl-ethyl]-benzenesulfonamide;
N-[2-(2,6-Dimethyl-phenoxy)-1-methyl-ethyl]-4-phenoxy-benzenesulfonamide;
4′-Fluoro-biphenyl-4-sulfonic acid [2-(2,6-dimethyl-phenoxy)-1-methyl-ethyl]-amide;
N-[2-(2,6-Dimethyl-phenoxy)-1-methyl-ethyl]-4-propyl-benzenesulfonamide;
N-[2-(2,6-Dimethyl-phenoxy)-1-methyl-ethyl]-4-(4-fluoro-phenoxy)-benzenesulfonamide;
N-[2-(2,6-Dimethyl-phenoxy)-1-methyl-ethyl]-4-(1,1-dimethyl-propyl)-benzenesulfonamide;
Naphthalene-2-sulfonic acid [2-(2,6-dimethyl-phenoxy)-1-methyl-ethyl]-amide;
Biphenyl-4-sulfonic acid [2-(2,6-dimethyl-phenoxy)-1-methyl-ethyl]-amide;
5-Bromo-thiophene-2-sulfonic acid [2-(2,6-dimethyl-phenoxy)-1-methyl-ethyl]-amide;
2-Bromo-N-[2-(2,6-dimethyl-phenoxy)-1-methyl-ethyl]-benzenesulfonamide;
N-[2-(2,6-Dimethyl-phenoxy)-1-methyl-ethyl]-3-methoxy-benzenesulfonamide;
4-n-Butoxy-N-[2-(2,6-dimethyl-phenoxy)-1-methyl-ethyl]-benzenesulfonamide;
N-[2-(2,6-Dimethyl-phenoxy)-1-methyl-ethyl]-4-(pyridin-2-yloxy)-benzenesulfonamide;
N-[2-(2,6-Dimethyl-phenoxy)-1-methyl-ethyl]-2,4,6-trimethyl-benzenesulfonamide;
N-[2-(2,6-Dimethyl-phenoxy)-1-methyl-ethyl]-3-p-tolyloxy-benzenesulfonamide;
4-Bromo-2-methyl-N-(2-phenoxy-ethyl)-benzenesulfonamide;
N-(2-Phenoxy-ethyl)-4-trifluoromethoxy-benzenesulfonamide;
4-(1,1-Dimethyl-propyl)-N-(2-phenoxy-ethyl)-benzenesulfonamide;
Biphenyl-4-sulfonic acid (2-phenoxy-ethyl)-amide;
2,4,6-Trimethyl-N-(2-phenoxy-ethyl)-benzenesulfonamide;
4-Bromo-N-(3-phenyl-propyl)-benzenesulfonamide;
4-Bromo-2-methyl-N-(3-phenyl-propyl)-benzenesulfonamide;
N-(3-Phenyl-propyl)-4-trifluoromethoxy-benzenesulfonamide;
4-Methoxy-2,3,6-trimethyl-N-(3-phenyl-propyl)-benzenesulfonamide;
4-tert-Butyl-N-(3-phenyl-propyl)-benzenesulfonamide;
4-Phenoxy-N-(3-phenyl-propyl)-benzenesulfonamide;
4′-Fluoro-biphenyl-4-sulfonic acid (3-phenyl-propyl)-amide;
N-(3-Phenyl-propyl)-4-propyl-benzenesulfonamide;
4-(4-Fluoro-phenoxy)-N-(3-phenyl-propyl)-benzenesulfonamide;
4-(1,1-Dimethyl-propyl)-N-(3-phenyl-propyl)-benzenesulfonamide;
Naphthalene-2-sulfonic acid (3-phenyl-propyl)-amide;
Biphenyl-4-sulfonic acid (3-phenyl-propyl)-amide;
5-Bromo-thiophene-2-sulfonic acid (3-phenyl-propyl)-amide;
2,4,6-Trimethyl-N-(3-phenyl-propyl)-benzenesulfonamide;
N-(3-Phenyl-propyl)-3-p-tolyloxy-benzenesulfonamide;
N-[(1S)-2-(5-Isoquinolinyloxy)-1-methylethyl]-2,4,6-trimethylbenzenesulfonamide;
N-[(1S)-2-(1H-Indol-4-yloxy)-1-methylethyl]-2,4,6-trimethylbenzenesulfonamide;
2,4,6-Trimethyl-N-[(1S)-1-methyl-2-(5-quinolinyloxy)ethyl]benzenesulfonamide;
N-[(1S)-2-(1,3-Benzodioxol-5-yloxy)-1-methylethyl]-2,4,6-trimethylbenzenesulfonamide;
2,4,6-Trimethyl-N-[(1S)-1-methyl-2-(4-quinolinyloxy)ethyl]benzenesulfonamide;
2,4,6-Trimethyl-N-[(1S)-1-methyl-2-(4-quinazolinyloxy)ethyl]benzenesulfonamide;
2,4,6-Trimethyl-N-[(1S)-1-methyl-2-(8-quinolinyloxy)ethyl]benzenesulfonamide;
5-Fluoro-2-({(2S)-2-[(mesitylsulfonyl)amino]propyl}oxy)benzamide;
2-({(2S)-2-[(Mesitylsulfonyl)amino]propyl}oxy)-5-methylbenzamide;
2-Hydroxy-6-({(2S)-2-[(mesitylsulfonyl)amino]propyl}oxy)benzamide;
5-Chloro-2-({(2S)-2-[(mesitylsulfonyl)amino]propyl}oxy)benzamide;
2-({(2S)-2-[(Mesitylsulfonyl)amino]propyl}oxy)-4-methylbenzamide;
2-({(2S)-2-[(Mesitylsulfonyl)amino]propyl}oxy)benzamide;
4-Fluoro-2-({(2S)-2-[(mesitylsulfonyl)amino]propyl}oxy)benzamide;
4-Chloro-2-({(2S)-2-[(mesitylsulfonyl)amino]propyl}oxy)benzamide;
5-Cyano-2-({(2S)-2-[(mesitylsulfonyl)amino]propyl}oxy)benzamide;
2-({(2S)-2-[(Mesitylsulfonyl)amino]propyl}oxy)-5-methoxybenzamide;
3-({(2S)-2-[(Mesitylsulfonyl)amino]propyl}oxy)-4-methylbenzamide;
2-({(2S)-2-[(Mesitylsulfonyl)amino]propyl}oxy)-4-methoxybenzamide;
2,5-Dichloro-N-[(1S)-2-(isoquinolin-5-yloxy)-1-methylethyl]thiophene-3-sulfonamide;
N-[(1S)-2-(Isoquinolin-5-yloxy)-1-methylethyl]-5-methyl-1-phenyl-1H-pyrazole-4-sulfonamide;
1-(Difluoromethyl)-N-[(1S)-2-(isoquinolin-5-yloxy)-1-methylethyl]-3,5-dimethyl-1H-pyrazole-4-sulfonamide;
N-[(1S)-2-(Isoquinolin-5-yloxy)-1-methylethyl]-2,5-dimethylfuran-3-sulfonamide;
2,5-Dichloro-N-[(1S)-1-methyl-2-(quinolin-5-yloxy)ethyl]thiophene-3-sulfonamide;
3-Bromo-5-chloro-N-[(1S)-1-methyl-2-(quinolin-5-yloxy)ethyl]thiophene-2-sulfonamide;
N-[(1S)-2-(Isoquinolin-5-yloxy)-1-methylethyl]-5-[1-methyl-5-(trifluoromethyl)-1H-pyrazol-3-yl]thiophene-2-sulfonamide;
1-(Difluoromethyl)-N-[(1S)-2-(isoquinolin-5-yloxy)-1-methylethyl]-5-methyl-1H-pyrazole-4-sulfonamide;
5-Methyl-N-[(1S)-1-methyl-2-(quinolin-5-yloxy)ethyl]-1-phenyl-1H-pyrazole-4-sulfonamide;
5-Chloro-N-[(1S)-2-(isoquinolin-5-yloxy)-1-methylethyl]thiophene-2-sulfonamide;
5-Chloro-N-[(1S)-1-methyl-2-(quinolin-5-yloxy)ethyl]thiophene-2-sulfonamide;
Methyl 4-({[(1S)-2-(isoquinolin-5-yloxy)-1-methylethyl]amino}sulfonyl)-2,5-dimethyl-3-furoate;
N-[(1S)-2-(Isoquinolin-5-yloxy)-1-methylethyl]thiophene-3-sulfonamide;
1-Ethyl-N-[(1S)-2-(isoquinolin-5-yloxy)-1-methylethyl]-1H-pyrazole-4-sulfonamide;
2-[((2S)-2-{[(2,5-Dichloro-3-thienyl)sulfonyl]amino}propyl)oxy]benzamide;
1-(Difluoromethyl)-3,5-dimethyl-N-[(1S)-1-methyl-2-(quinolin-5-yloxy)ethyl]-1H-pyrazole-4-sulfonamide;
N-[(1S)-1-Methyl-2-(quinolin-5-yloxy)ethyl]-5-[1-methyl-5-(trifluoromethyl)-1H-pyrazol-3-yl]thiophene-2-sulfonamide;
1-Ethyl-N-[(1S)-1-methyl-2-(quinolin-5-yloxy)ethyl]-1H-pyrazole-4-sulfonamide;
2-({(2S)-2-[({5-[1-Methyl-5-(trifluoromethyl)-1H-pyrazol-3-yl]-2-thienyl}sulfonyl)-amino]propyl}oxy)benzamide;
2-[((2S)-2-{[(2,5-Dimethyl-3-thienyl)sulfonyl]amino}propyl)oxy]benzamide;
2,5-Dimethyl-N-[(1S)-1-methyl-2-(quinolin-5-yloxy)ethyl]furan-3-sulfonamide;
2-[((2S)-2-{[(2,5-Dimethyl-3-furyl)sulfonyl]amino}propyl)oxy]benzamide;
2-{[(2S)-2-({[1-(Difluoromethyl)-3,5-dimethyl-1H-pyrazol-4-yl]sulfonyl}amino)propyl]-oxy}benzamide;
1-Ethyl-N-[(1S)-2-(isoquinolin-5-yloxy)-1-methylethyl]-3-methyl-1H-pyrazole-4-sulfonamide;
N-[(1S)-2-(Isoquinolin-5-yloxy)-1-methylethyl]-1,3,5-trimethyl-1H-pyrazole-4-sulfonamide;
N-[(1S)-2-(Isoquinolin-5-yloxy)-1-methylethyl]-3,5-dimethylisoxazole-4-sulfonamide;
N-[(1S)-2-(Isoquinolin-5-yloxy)-1-methylethyl]-2,5-dimethylthiophene-3-sulfonamide;
2,4,6-Trimethyl-N-{(1S)-1-methyl-2-[(8-methylquinolin-5-yl)amino]ethyl}-benzenesulfonamide;
2,4,6-Trimethyl-N-{(1S)-1-methyl-2-[(6-methylquinolin-5-yl)amino]ethyl}-benzenesulfonamide;
N-[(1S)-2-(1H-Indazol-4-ylamino)-1-methylethyl]-2,4,6-trimethylbenzenesulfonamide;
2,4,6-Trimethyl-N-[(1S)-1-methyl-2-(quinolin-5-ylamino)ethyl]benzenesulfonamide;
N-[(1S)-2-(1H-Indazol-6-ylamino)-1-methylethyl]-2,4,6-trimethylbenzenesulfonamide;
2,4,6-Trimethyl-N-{(1S)-1-methyl-2-[(2-methylquinolin-5-yl)amino]ethyl}-benzenesulfonamide;
N-[(1S)-2-(1H-Indazol-5-ylamino)-1-methylethyl]-2,4,6-trimethylbenzenesulfonamide;
N-((1S)-2-{[2-Chloro-4-(methylsulfonyl)phenyl]amino}-1-methylethyl)-2,4,6-trimethylbenzenesulfonamide;
N-[(1S)-2-(4-Cyano-2,6-dimethylphenoxy)-1-methylethyl]-2,4,6-trimethylbenzenesulfonamide;
N-[(1S)-2-(3-Cyanophenoxy)-1-methylethyl]-2,4,6-trimethylbenzenesulfonamide;
N-[(1S)-2-(3-Methoxyphenoxy)-1-methylethyl]-2,4,6-trimethylbenzenesulfonamide;
N-[2-(3,5-Dimethoxyphenoxy)-1-methylethyl]-2,4,6-trimethylbenzenesulfonamide;
N-[2-(4-Cyano-2-methoxyphenoxy)-1-methylethyl]-2,4,6-trimethylbenzenesulfonamide;
N-{2-[(2-Bromopyridin-3-yl)oxy]-1-methylethyl}-2,4,6-trimethylbenzenesulfonamide;
2,4,6-Trimethyl-N-{1-methyl-2-[(2-methylpyridin-3-yl)oxy]ethyl}benzenesulfonamide;
2-{2-[(Mesitylsulfonyl)amino]propoxy}-N-methylbenzamide;
4-{2-[(Mesitylsulfonyl)amino]propoxy}benzamide;
N-{2-[4-(1H-Imidazol-1-yl)phenoxy]-1-methylethyl}-2,4,6-trimethylbenzenesulfonamide;
N-[(1S)-2-(3,4-Dimethoxyphenoxy)-1-methylethyl]-2,4,6-trimethylbenzenesulfonamide;
N-(2-{2-[(Mesitylsulfonyl)amino]propoxy}phenyl)acetamide;
N-{2-[(6-Chloropyridin-3-yl)oxy]-1-methylethyl}-2,4,6-trimethylbenzenesulfonamide;
N-[(1S)-2-(2H-Indazol-3-yloxy)-1-methylethyl]-2,4,6-trimethylbenzenesulfonamide;
4-Methyl-N-[3-phenyl-1-(trifluoromethyl)propyl]benzenesulfonamide;
N-[(1S)-2-(Isoquinolin-5-yloxy)-1-methylethyl]-2,4-dimethylbenzenesulfonamide;
N-[(1S)-2-(Isoquinolin-5-yloxy)-1-methylethyl]-3,4-dimethylbenzenesulfonamide;
N-[(1S)-2-(Isoquinolin-5-yloxy)-1-methylethyl]-2,5-dimethylbenzenesulfonamide;
2,4-Dimethyl-N-[(1S)-1-methyl-2-(quinolin-5-yloxy)ethyl]benzenesulfonamide;
3,4-Dimethyl-N-[(1S)-1-methyl-2-(quinolin-5-yloxy)ethyl]benzenesulfonamide;
2-[((2S)-2-{[(2,4-Dimethylphenyl)sulfonyl]amino}propyl)oxy]benzamide;
2,5-Dimethyl-N-[(1S)-1-methyl-2-(quinolin-5-yloxy)ethyl]benzenesulfonamide;
2-[((2S)-2-{[(3,4-Dimethylphenyl)sulfonyl]amino}propyl)oxy]benzamide;
N-(2-Anilinoethyl)-2,4,6-trimethylbenzenesulfonamide;
N-[2-(2,6-Dimethylphenoxy)-1-methylethyl]-4-(trifluoromethyl)benzenesulfonamide;
N-(2-Anilinoethyl)-4′-fluorobiphenyl-4-sulfonamide;
N-(2-Anilinoethyl)-4-methoxy-2,3,6-trimethylbenzenesulfonamid;
N-(2-Anilinoethyl)-4-bromo-2-methylbenzenesulfonamid;
1-(4-Fluorophenyl)-N-[(1S)-2-(isoquinolin-5-yloxy)-1-methylethyl]-3,5-dimethyl-1H-pyrazole-4-sulfonamide;
N-[(1S)-2-(Isoquinolin-5-yloxy)-1-methylethyl]-3,5-dimethyl-1-phenyl-1H-pyrazole-4-sulfonamide;
N,2,4,6-Tetramethyl-N-[(1S)-1-methyl-3-phenylpropyl]benzenesulfonamide;
2,4,6-Trimethyl-N-{1-[(quinolin-5-yloxy)methyl]propyl}benzenesulfonamide;
5-Chloro-2-{2-[(mesitylsulfonyl)amino]butoxy}benzamide;
2,4-Dichloro-6-methyl-N-[(1S)-1-methyl-2-(quinolin-5-yloxy)ethyl]benzenesulfonamide;
5-Chloro-2-{[(2S)-2-({[4-(4-fluorophenoxy)phenyl]sulfonyl}amino)propyl]oxy}benzamide;
5-Chloro-2-{[(2S)-2-({[4-(4-methoxyphenoxy)phenyl]sulfonyl}amino)propyl]oxy}-benzamide;
5-Chloro-2-{[(2S)-2-({[3-(4-chlorophenoxy)phenyl]sulfonyl}amino)propyl]oxy}benzamide;
2,4,5-Trichloro-N-[(1S)-1-methyl-2-(quinolin-5-yloxy)ethyl]benzenesulfonamide;
5-Chloro-2-{[(2S)-2-({[3-(3,4-dichlorophenoxy)phenyl]sulfonyl}amino)propyl]oxy}-benzamide;
3-(4-Chlorophenoxy)-N-[(1S)-1-methyl-2-(quinolin-5-yloxy)ethyl]benzenesulfonamide;
5-Chloro-2-[((2S)-2-{[(2,4-dichloro-5-fluorophenyl) sulfonyl]amino}propyl)oxy]benzamide;
5-Chloro-2-{[(2S)-2-({[3-(4-methoxyphenoxy)phenyl]sulfonyl}amino)propyl]oxy}benzamide;
5-Chloro-2-[((2S)-2-{[(2-methoxy-4-methylphenyl)sulfonyl]amino}propyl)oxy]benzamide;
4-(4-Fluorophenoxy)-N-[(1S)-1-methyl-2-(quinolin-5-yloxy)ethyl]benzenesulfonamide;
5-Chloro-2-[((2S)-2-{[(5-chloro-2-methoxyphenyl)sulfonyl]amino}propyl)oxy]benzamide;
3-Cyano-N-[(1S)-1-methyl-2-(quinolin-5-yloxy)ethyl]benzenesulfonamide;
2,4-Dichloro-5-fluoro-N-[(1S)-1-methyl-2-(quinolin-5-yloxy)ethyl]benzenesulfonamide;
2-[((2S)-2-{[(5-Bromo-2-methoxyphenyl)sulfonyl]amino}propyl)oxy]-5-chlorobenzamide;
5-Chloro-2-[((2S)-2-{[(2-methoxy-5-methylphenyl)sulfonyl]amino}propyl)oxy]benzamide;
5-Chloro-2-{[(2S)-2-({[4′-(trifluoromethyl)biphenyl-4-yl]sulfonyl}amino)propyl]oxy}-benzamide;
4-(4-Methoxyphenoxy)-N-[(1S)-1-methyl-2-(quinolin-5-yloxy)ethyl]benzenesulfonamide;
5-Chloro-2-[((2S)-2-{[(6-phenoxypyridin-3-yl)sulfonyl]amino}propyl)oxy]benzamide;
5-Bromo-6-chloro-N-[(1S)-1-methyl-2-(quinolin-5-yloxy)ethyl]pyridine-3-sulfonamide;
5-Bromo-2-methoxy-N-[(1S)-1-methyl-2-(quinolin-5-yloxy)ethyl]benzenesulfonamide;
N-[(1S)-1-Methyl-2-(quinolin-5-yloxy)ethyl]-1-benzothiophene-2-sulfonamide;
5-Chloro-2-[((2S)-2-{[(2,4-dimethoxyphenyl)sulfonyl]amino}propyl)oxy]benzamide;
2-({(2S)-2-[(1-Benzothien-2-ylsulfonyl)amino]propyl}oxy)-5-chlorobenzamide;
5-Chloro-2-[((2S)-2-{[(4-methoxy-2,3,6-trimethylphenyl)sulfonyl]amino}propyl)oxy]-benzamide;
5-Chloro-2-[((2S)-2-{[(5-fluoro-3-methyl-1-benzothien-2-yl)sulfonyl]amino}propyl)oxy]-benzamide;
5-Chloro-2-[((2S)-2-{[(5-chloro-3-methyl-1-benzothien-2-yl)sulfonyl]amino}propyl)oxy]-benzamide;
2-{[(2S)-2-({[4-Bromo-2-(trifluoromethoxy)phenyl]sulfonyl}amino)propyl]oxy}-5-chlorobenzamide;
2,4,6-Trichloro-N-[(1S)-1-methyl-2-(quinolin-5-yloxy)ethyl]benzenesulfonamide;
4-Methoxy-2,3,6-trimethyl-N-[(1S)-1-methyl-2-(quinolin-5-yloxy)ethyl]-benzenesulfonamide; or,
4-Bromo-N-[(1S)-1-methyl-2-(quinolin-5-yloxy)ethyl]-2-(trifluoromethoxy)-benzenesulfonamide;
   or a pharmaceutically acceptable salt thereof.

The compounds of formula (I) can be prepared using or adapting methods disclosed in the art, or by using or adapting the method disclosed in the Examples below. Starting materials for the preparative methods are either commercially available or can be prepared by literature methods, adapting literature methods.

For example, a compound of the invention can be prepared by coupling a compound of formula (II):

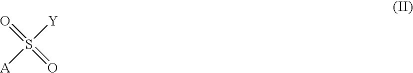

wherein Y is a leaving group (for example chlorine), with a compound of formula (III):

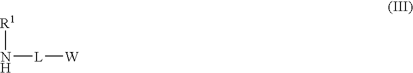

in a suitable solvent (such as tetrahydrofuran or N,N-dimethylformamide) at a temperature in the range −10° C. to 50° C.

The invention further provides processes for the preparation of the compounds of formula (I).

Because of their ability to bind to the glucocorticoid receptor the compounds of formula (I) are useful as anti-inflammatory agents, and can also display antiallergic, immunosuppressive and anti-proliferative actions. Thus, a compound of formula (I), or a pharmaceutically acceptable salt thereof can be used as a medicament for the treatment or prophylaxis of one or more of the following pathologic conditions (disease states) in a mammal (such as a human):
(i) Lung diseases, which coincide with inflammatory, allergic and/or proliferative processes:
   chronically obstructive lung diseases of any origin, mainly bronchial asthma
   bronchitis of different origins
   all forms of restructive lung diseases, mainly allergic alveolitis
   all forms of pulmonary edema, mainly toxic pulmonary edema
   sarcoidoses and granulomatoses, such as Boeck's disease
(ii) Rheumatic diseases/auto-immune diseases/degenerative joint diseases, which coincide with inflammatory, allergic and/or proliferative processes:
   all forms of rheumatic diseases, especially rheumatoid arthritis, acute rheumatic fever, polymyalgia rheumatica, collagenoses
   reactive arthritis
   inflammatory soft-tissue diseases of other origins
   arthritic symptoms in degenerative joint diseases (arthroses)
   traumatic arthritides
   collagen diseases of other origins, for example systemic lupus erythematodes, sclerodermia, polymyositis, dermatomyositis, polyarteritis nodosa, temporal arteritis
   Sjögren's syndrome, Still syndrome, Felty's syndrome
(iii) Allergies, which coincide with inflammatory, allergic and/or proliferative processes:
   All forms of allergic reactions, for example Quincke's edema, hay fever, insect bites, allergic reactions to pharmaceutical agents, blood derivatives, contrast media, etc., anaphylactic shock, urticaria, contact dermatitis
(iv) Dermatological diseases, which coincide with inflammatory, allergic and/or proliferative processes:
   atopic dermatitis (mainly in children)
   psoriasis
   erythematous diseases, triggered by different noxae, for example radiation, chemicals, burns, etc.
   acid burns
   bullous dermatoses
   diseases of the lichenoid group
   itching (for example of allergic origins)
   seborrheal eczema
   rosacea
   pemphigus vulgaris
   erythema exudativum multiforme
   erythema nodosum
   balanitis
   vulvitis
   inflammatory hair loss, such as alopecia areata
   cutaneous T-cell lymphoma
(v) Nephropathies, which coincide with inflammatory, allergic and/or proliferative processes:
   nephrotic syndrome
   all nephritides
(vi) Liver diseases, which coincide with inflammatory, allergic and/or proliferative processes:
   acute liver cell decomposition
   acute hepatitis of different origins, for example virally-, toxically- or pharmaceutical agent-induced
   chronically aggressive and/or chronically intermittent hepatitis
(vii) Gastrointestinal diseases, which coincide with inflammatory, allergic and/or proliferative processes:
   regional enteritis (Crohn's disease)
   ulcerative colitis
   gastroenteritis of other origins, for example native sprue
(viii) Proctological diseases, which coincide with inflammatory, allergic and/or proliferative processes:
   anal eczema
   fissures
   haemorrhoids
   idiopathic proctitis
(ix) Eve diseases, which coincide with inflammatory, allergic and/or proliferative processes:
   allergic keratitis, uvenitis iritis
   conjunctivitis
   blepharitis
   optic neuritis
   chorioiditis
   sympathetic ophthalmia
(x) Diseases of the ear-nose-throat area, which coincide with inflammatory, allergic and/or proliferative processes:
   allergic rhinitis, hay fever
   otitis externa, for example caused by contact dermatitis, infection, etc.
   otitis media
(xi) Neurological diseases, which coincide with inflammatory, allergic and/or proliferative processes:
   cerebral edema, mainly tumor-induced cerebral edema
   multiple sclerosis
   acute encephalomyelitis
   different forms of convulsions, for example infantile nodding spasms
(xii) Blood diseases, which coincide with inflammatory, allergic and/or proliferative processes:
   acquired haemolytic anemia
   idiopathic thrombocytopenia
(xiii) Tumor diseases, which coincide with inflammatory, allergic and/or proliferative processes:
   acute lymphatic leukaemia
   malignant lymphoma
   lymphogranulomatoses
   lymphosarcoma
   extensive metastases, mainly in breast and prostate cancers
(xiv) Endocrine diseases, which coincide with inflammatory, allergic and/or proliferative processes:
   endocrine orbitopathy
   thyrotoxic crisis
   de Quervain's thyroiditis
   Hashimoto's thyroiditis
   hyperthyroidism
(xv) Transplants, which coincide with inflammatory, allergic and/or proliferative processes;
(xvi) Severe shock conditions, which coincide with inflammatory, allergic and/or proliferative processes, for example anaphylactic shock
(xvii) Substitution therapy, which coincides with inflammatory, allergic and/or proliferative processes, with:
   innate primary suprarenal insufficiency, for example congenital adrenogenital syndrome
   acquired primary suprarenal insufficiency, for example Addison's disease, autoimmune adrenalitis, meta-infective, tumors, metastases, etc.
   innate secondary suprarenal insufficiency, for example congenital hypopituitarism
   acquired secondary suprarenal insufficiency, for example meta-infective, tumors, etc.
(xviii) Emesis, which coincides with inflammatory, allergic and/or proliferative processes:
   for example in combination with a 5-HT₃-antagonist in cytostatic-agent-induced vomiting.

Without prejudice to the foregoing, the compounds of formula (I) can also be used to treat disorders such as: Conies Syndrome, primary and secondary hyperaldosteronism, increased sodium retention, increased magnesium and potassium excretion (diuresis), increased water retention, hypertension (isolated systolic and combined systolic/diastolic), arrhythmias, myocardial fibrosis, myocardial infarction, Bartter's Syndrome, disorders associated with excess catecholamine levels, diastolic and systolic congestive heart failure (CHF), peripheral vascular disease, diabetic nephropathy, cirrhosis with edema and ascites, oesophageal varicies, Addison's Disease, muscle weakness, increased melanin pigmentation of the skin, weight loss, hypotension, hypoglycemia, Cushing's Syndrome, obesity, hypertension, glucose intolerance, hyperglycemia, diabetes mellitus, osteoporosis, polyuria, polydipsia, inflammation, autoimmune disorders, tissue rejection associated with organ transplant, malignancies such as leukemias and lymphomas, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia, and Little's syndrome, systemic inflammation, inflammatory bowel disease, systemic lupus erythematosus, discoid lupus erythematosus, polyartitis nodosa, Wegener's granulomatosis, giant cell arthritis, rheumatoid arthritis, osteoarthritis, hay fever, allergic rhinitis, contact dermatitis, atopic dermatitis, exfoliative dermatitis, urticaria, angioneurotic edema, chronic obstructive pulmonary disease, asthma, tendonitis, bursitis, Crohn's disease, ulcerative colitis, autoimmune chronic active hepatitis, hepatitis, cinhosis, inflammatory scalp alopecia, panniculitis, psoriasis, inflamed cysts, pyoderma gangrenosum, pemphigus vulgaris, bullous pemphigoid, dermatomyositis, eosinophilic fasciitis, relapsing polychondritis, inflammatory vasculitis, sarcoidosis Sweet's disease, type 1 reactive leprosy, capillary hemangiomas, lichen planus, erythema nodosum acne, hirsutism, toxic epidermal necrolysis, erythema multiform, cutaneous T-cell lymphoma, psychoses, cognitive disorders (such as memory disturbances) mood disorders (such as depression and bipolar disorder), anxiety disorders and personality disorders.

As used herein the term “congestive heart failure” (CHF) or “congestive heart disease” refers to a disease state of the cardiovascular system whereby the heart is unable to efficiently pump an adequate volume of blood to meet the requirements of the body's tissues and organ systems. Typically, CHF is characterized by left ventricular failure (systolic dysfunction) and fluid accumulation in the lungs, with the underlying cause being attributed to one or more heart or cardiovascular disease states including coronary artery disease, myocardial infarction, hypertension, diabetes, valvular heart disease, and cardiomyopathy. The term “diastolic congestive heart failure” refers to a state of CHF characterized by impairment in the ability of the heart to properly relax and fill with blood. Conversely, the term “systolic congestive heart failure” refers to a state of CHF characterized by impairment in the ability of the heart to properly contract and eject blood.

As will be appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of physiological disorders contemplates both acute events and chronic conditions. In an acute event, compound is administered at the onset of symptoms and discontinued when the symptoms disappear.

In another aspect the present invention provides the use of a compound or formula (I), or a pharmaceutically acceptable salt thereof, for use in therapy (such as a therapy described above).

In yet another aspect the present invention provides the use of a compound or formula (I), or a pharmaceutically acceptable salt thereof, in the manufacture of a medicament for use in the treatment of a glucocorticoid receptor mediated disease state (such as a disease state described above).

In a further aspect the invention provides the use of a compound of formula (I), or a pharmaceutically acceptable salt thereof, in the manufacture of a medicament for use in the treatment of an inflammatory (such as an arthritic) condition.

In a still further aspect the invention provides the use of a compound of formula (I), or a pharmaceutically acceptable salt thereof, in the manufacture of a medicament for use in the treatment of an asthmatic or dermatological condition.

In another aspect the invention provides the use of a compound of formula (I), or a pharmaceutically acceptable salt thereof, in the manufacture of a medicament for use in the treatment of COPD.

The present invention further provides a method of treating a glucocorticoid receptor mediated disease state in a mammal (such as man), which comprises administering to a mammal in need of such treatment an effective amount of a compound of formula (I), or a pharmaceutically acceptable salt thereof.

In order to use a compound of formula (I), or a pharmaceutically acceptable salt thereof, for the therapeutic treatment of a mammal, said active ingredient is normally formulated in accordance with standard pharmaceutical practice as a pharmaceutical composition.

Therefore in another aspect the present invention provides a pharmaceutical composition comprising a compound of formula (I), or a pharmaceutically acceptable salt thereof, (active ingredient) and a pharmaceutically acceptable adjuvant, diluent or carrier. In a further aspect the present invention provides a process for the preparation of said composition comprising mixing the active ingredient with a pharmaceutically acceptable adjuvant, diluent or carrier. Depending on the mode of administration, the pharmaceutical composition can comprise from 0.05 to 99% w (percent by weight), for example from 0.05 to 80% w, such as from 0.10 to 70% w (for example from 0.10 to 50% w), of active ingredient, all percentages by weight being based on total composition.

A pharmaceutical composition of the present invention can be administered in a standard manner for the disease condition that it is desired to treat, for example by topical (such as to the lung and/or airways or to the skin), oral, rectal or parenteral administration. Thus, a the compound of formula (I), or a pharmaceutically acceptable salt thereof, may be formulated into the form of, for example, an aerosol, a powder (for example dry or dispersible), a tablet, a capsule, a syrup, a granule, an aqueous or oily solution or suspension, an (lipid) emulsion, a suppository, an ointment, a cream, drops, or a sterile injectable aqueous or oily solution or suspension.

A suitable pharmaceutical composition of this invention is one suitable for oral administration in unit dosage form, for example a tablet or capsule containing between 0.1 mg and 1 g of active ingredient.

In another aspect a pharmaceutical composition of the invention is one suitable for intravenous, subcutaneous, intraarticular or intramuscular injection.

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. Tablets may be enteric coated by conventional means, for example to provide a coating of cellulose acetate phthalate.

The invention further relates to combination therapies or compositions wherein a compound of formula (I), or a pharmaceutically acceptable salt thereof, or a pharmaceutical composition comprising a compound of formula (I), or a pharmaceutically acceptable salt thereof, is administered concurrently (possibly in the same composition) or sequentially with an agent for the treatment of any one of the above disease states.

In particular, for the treatment of the inflammatory diseases (for example rheumatoid arthritis, COPD, asthma or allergic rhinitis) a compound of the invention can be combined with a TNF-α inhibitor (such as an anti-TNF monoclonal antibody (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:
a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;
a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;
a PDE4 inhibitor including an inhibitor of the isoform PDE4D;
an antihistaminic H.sub1. receptor antagonist such as cetirizine, loratadine, desloratadine, fexofenadine, astemizole, azelastine or chlorpheniramine;
a gastroprotective H.sub2. receptor antagonist;
an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;
an anticholinergic agent such as ipratropium bromide, tiotropium bromide, oxitropium bromide, pirenzepine or telenzepine;
a β.sub1.- to β.sub4.-adrenoceptor agonist (such as β2 adrenoceptor agonist) such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;
an insulin-like growth factor type I (IGF-1) mimetic;
an inhaled glucocorticoid with reduced systemic side effects, such as prednisone, prednisolone, flunisolide, triamcinolone acetonide, beclomethasone dipropionate, budesonide, fluticasone propionate or mometasone furoate;
an inhibitor of a matrix metalloprotease (MMP), such as a stromelysin, a collagenase, or a gelatinase or aggrecanase; such as collagenase-1 (MMP-1), collagenase-2 (MMP-8), collagenase-3 (MMP-13), stromelysin-1 (MMP-3), stromelysin-2 (MMP-10), and stromelysin-3 (MMP-11) or MMP-12;
a modulator of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family;
an osteoporosis agent such as roloxifene, droloxifene, lasofoxifene or fosomax;
an immunosuppressant agent such as FK-506, rapamycin, cyclosporine, azathioprine or methotrexate;
a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group 120 antibody (or modified/recombinant antibody); or another agent which interferes with the binding of group 120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfmavir (for example as free base or as mesylate salt), amprenavir, lopinavir or atazanavir (for example as free base or as sulphate salt)}; a ribonucleotide reductase inhinbitor {for example hydroxyurea}; or an antiretroviral {for example emtricitabine}; or,
an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The following compounds illustrate compounds of formula (I)

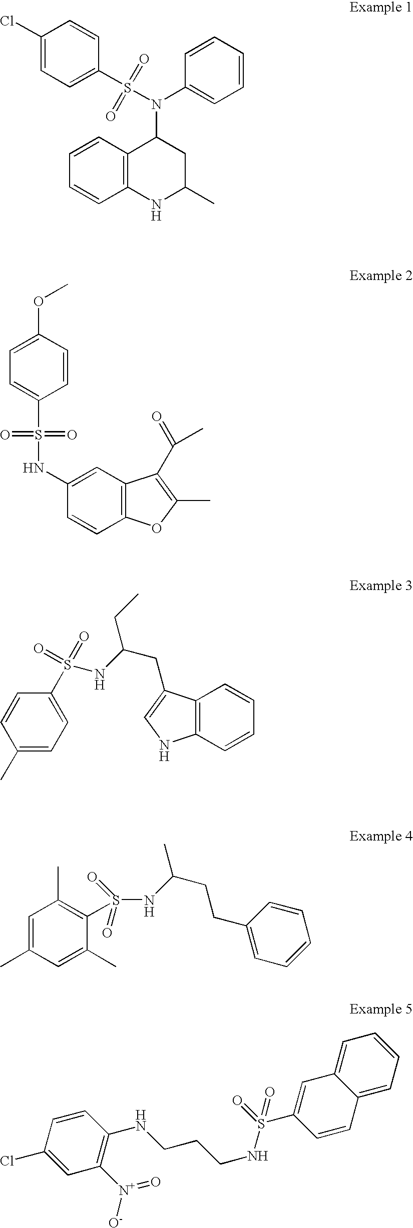

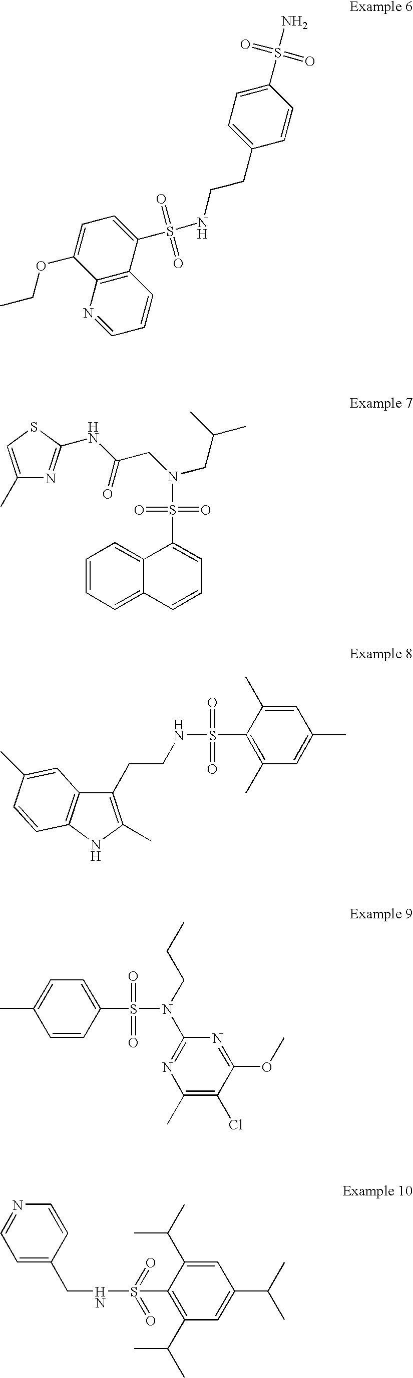

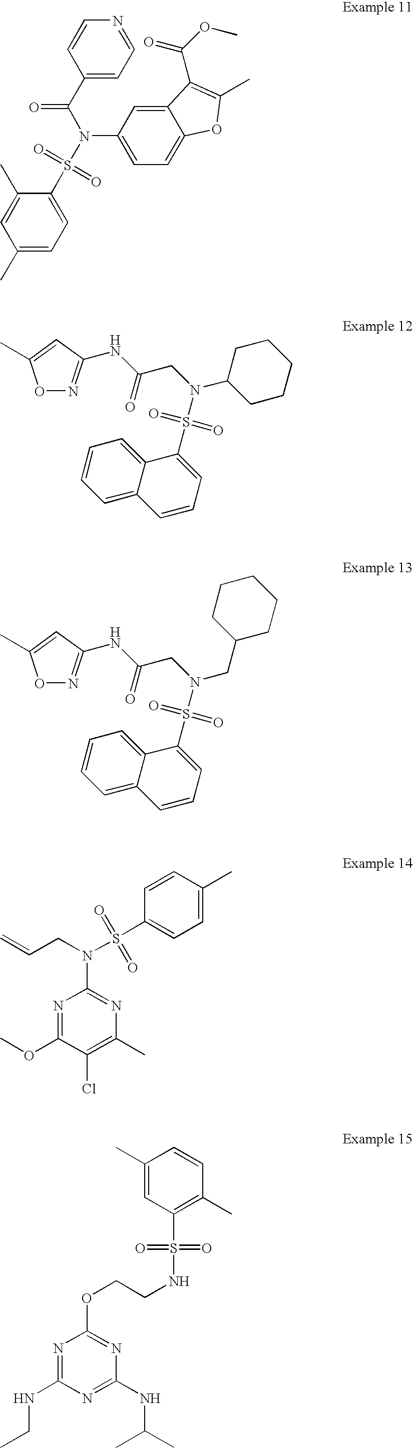

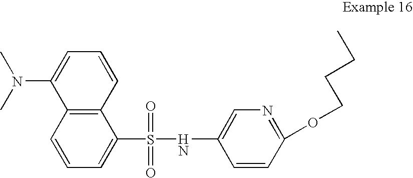

The following abbreviations are used in the following preparative Examples:
THF tetrahydrofuran
TFA trifluoroacetic acid
DMSO dimethylsulfoxide
DMF N,N-dimethylformamide
TBAT N,N,N-tributylbutan-1-aminium difluoro(triphenyl)silicate
DIEA diisopropylethyl amine
NMP 1-Methyl-2-pyrrolidinone
app approximately
sat saturated
aq aqueous

### General Methods

¹H NMR spectra were recorded on a Varian Mercury-VX 300 MHz instrument or aVarian Unity 400 MHz instrument. The central peaks of chloroform-d (δ_{H} 7.27 ppm), acetonitrile-d3 (δ_{H} 1.95 ppm), or DMSO-d6 (δ_{H} 2.50 ppm) were used as internal references. Low resolution mass spectra and accurate mass determination were recorded on a Hewlett-Packard 1100 LC-MS system equipped with APCI ionisation chamber. Unless stated otherwise, starting materials were commercially available. All solvents and commercial reagents were of laboratory grade and were used as received.

The following methods were used for LC/MS analysis

Method A: Instrument Agilent 1100; Column C₁₈ Waters Symmetry 2.1×30 mm 3.5 μm; Flow rate 0.7 ml/min; Mass APCI; UV-absorption was measured at 254 nm; Solvent A: water+0.1% TFA; Solvent B: acetonitrile+0.1% TFA; Gradient 5-95%/B 8 min, 95% B 2 min.
Method B: Instrument Agilent 1100; Column Kromasil C₁₈ 3×100 mm 5 μm; Flow rate 1.0 ml/min; UV-absorption was measured at 254 nm; Solvent A: water+0.1% TFA; Solvent B: acetonitrile+0.1% TFA; Gradient 10-100% B 20 min, 100% B 1 min.

### EXAMPLE 17

### 4-Bromo-N-(1-methyl-3-phenyl-propyl)-benzenesulfonamide

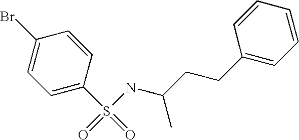

4-Bromo-benzenesulfonyl chloride (120 μL 0.3M/THF) was mixed with 1-methyl-3-phenyl-propylamine (100 μL 0.3M/pyridine) and stirred overnight in ambient temperature before it was evaporated to dryness under reduced pressure. The residue was purified on HPLC-C₁₈ yielding 2.1 mg (25%).

¹H NMR (299.944 MHz, CDCl₃) δ 7.68 (ddt, J=23.9, 8.8, 2.1 Hz, 3H), 7.30-7.15 (m, 3H), 7.06 (dd, J=6.7, 1.6 Hz, 2H), 4.48 (d, J=5.9 Hz, 1H), 3.35 (q, J=6.2 Hz, 1H), 2.57 (ddd, J=29.9, 14.0, 7.9 Hz, 3H), 1.71 (td, J=7.8, 6.6 Hz, 2H), 1.10 (d, J=6.6 Hz, 3H) LC (method A) rt=6.1 min. UV 254 nm

Examples 18-76 were synthesised by a method analogous to that described in Example 17 using the corresponding starting materials.

### EXAMPLE 18

### 4-Chloro-N-(1-methyl-3-phenyl-propyl)-benzenesulfonamide

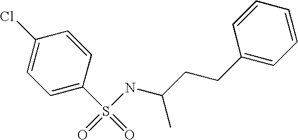

¹H NMR (299.944 MHz, CDCl₃) δ 7.79 (dt, J=9.0, 2.2 Hz, 2H), 7.47 (dt, J=8.9, 2.2 Hz, 2H), 7.30-7.17 (m, 3H), 7.06 (d, J=6.8 Hz, 2H), 4.46 (d, J=7.7 Hz, 1H), 3.37 (quintet, J=6.7 Hz, 1H), 2.57 (ddd, J=29.9, 14.0, 7.8 Hz, 2H), 1.71 (td, J=7.8, 6.6 Hz, 2H), 1.10 (d, J=6.6 Hz, 3H)

LC (method A) rt=6.0 min. UV 254 nm.

### EXAMPLE 19

### 4-Bromo-2-methyl-N-(1-methyl-3-phenyl-propyl)-benzenesulfonamide

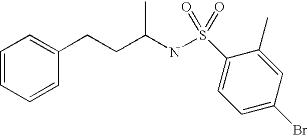

¹H NMR (299.944 MHz, CDCl₃) δ 7.82 (d, J=8.3 Hz, 1H), 7.50-7.42 (m, 2H), 7.28-7.16 (m, 3H), 7.03-7.00 (m, 2H), 4.48 (s, 1H), 3.31 (d, J=5.5 Hz, 1H), 2.63 (s, 3H), 2.61-2.45 (m, 2H), 1.76-1.64 (m, 2H), 1.11 (d, J=6.4 Hz, 3H)

LC (method A) rt=6.5 min. UV 254 nm.

### EXAMPLE 20

### N-(1-Methyl-3-phenyl-propyl)-4-trifluoromethoxy-benzenesulfonamide

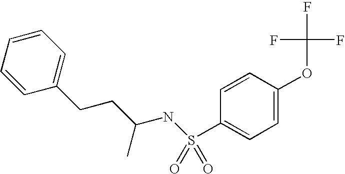

LC (method A) rt=6.3 min. UV 254 nm.

### EXAMPLE 21

### 4-Methoxy-2,3,6-trimethyl-N-(1-methyl-3-phenyl-propyl)-benzenesulfonamide

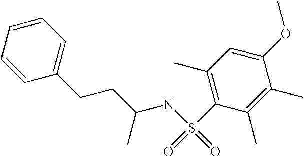

¹H NMR (299.944 MHz, CDCl₃) δ 7.26-7.12 (m, 3H), 7.02-6.97 (m, 2H), 6.58 (s, 1H), 3.87 (s, 3H), 3.30 (q, J=6.5 Hz, 1H), 2.65 (s, 3H), 2.59 (s, 4H), 2.57-2.43 (m, 6H), 2.16 (s, 3H), 1.73-1.63 (m, 2H), 1.10 (d, J=6.6 Hz, 3H)

APCI-MS m/z: 362.2 [MH+].

LC (method A) rt=6.4 min. UV 254 nm.

### EXAMPLE 22

### 4-tert-Butyl-N-(1-methyl-3-phenyl-propyl)-benzenesulfonamide

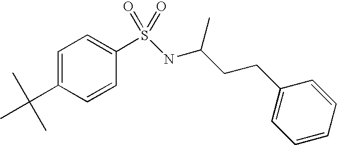

¹H NMR (299.944 MHz, CDCl₃) δ 7.83 (dd, J=6.8, 1.8 Hz, 2H), 7.54 (dd, J=6.8, 1.8 Hz, 2H), 7.30-7.17 (m, 3H), 7.06 (d, J=6.6 Hz, 2H), 4.49 (d, J=8.1 Hz, 1H), 3.42 (quintet, J=6.8 Hz, 1H), 2.58 (dtd, J=21.9, 14.1, 7.9 Hz, 2H), 1.75-1.67 (m, 2H), 1.38 (s, 9H), 1.12 (d, J=6.6 Hz, 3H)

APCI-MS m/z: 346.3 [MH+].

LC (method A) rt=6.6 min. UV 254 nm.

### EXAMPLE 23

### N-(1-Methyl-3-phenyl-propyl)-4-phenoxy-benzenesulfonamide

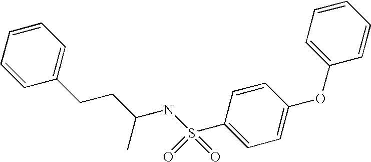

APCI-MS m/z: 382.1 [MH+].

LC (method A) rt=6.6 min. UV 254 nm.

### EXAMPLE 24

### 4′-Fluoro-biphenyl-4-sulfonic acid (1-methyl-3-phenyl-propyl)-amide

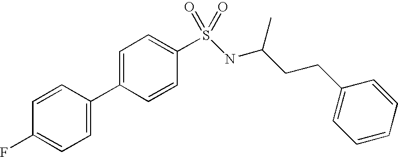

¹H NMR (299.944 MHz, CDCl₃) δ 8.01 (dd, J=6.7, 1.9 Hz, 2H), 7.75 (dd, J=6.7, 1.7 Hz, 2H), 7.70-7.64 (m, 2H), 7.35-7.23 (m, 5H), 7.15-7.13 (m, 2H), 4.52 (s, OH), 3.52 (q, J=6.4 Hz, 1H), 2.67 (ddd, J=32.7, 14.0, 7.9 Hz, 3H), 1.81 (dd, J=14.5, 7.9 Hz, 2H), 1.21 (d, J=6.6 Hz, 3H)

LC (method A) rt=6.6 min. UV 254 nm.

### EXAMPLE 25

### N-(1-Methyl-3-phenyl-propyl)-4-propyl-benzenesulfonamide

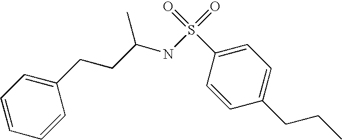

APCI-MS m/z: 332.2 [MH+].

LC (method A) rt=6.5 min. UV 254 nm.

### EXAMPLE 26

### N-(1-Methyl-3-phenyl-propyl)-4-trifluoromethyl-benzenesulfonamide

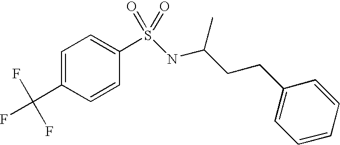

¹H NMR (299.944 MHz, CDCl₃) δ 7.99 (d, J=8.1 Hz, 2H), 7.78 (d, J=8.3 Hz, 2H), 7.30-7.18 (m, 3H), 7.06-7.04 (m, 2H), 4.57 (d, J=8.4 Hz, 1H), 3.42 (dt, J=14.9, 6.6 Hz, 1H), 2.59 (ddd, J=29.1, 13.9, 7.6 Hz, 2H), 1.77-1.70 (m, 2H), 1.13 (d, J=6.4 Hz, 3H)

LC (method A) rt=6.2 min. UV 254 nm.

### EXAMPLE 27

### 4-(1,1-Dimethyl-propyl)-N-(1-methyl-3-phenyl-propyl)-benzenesulfonamide

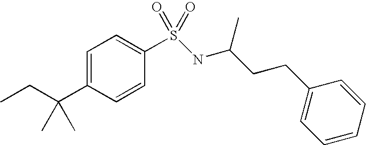

APCI-MS m/z: 360.2 [MH+].

LC (method A) rt=7.2 min. UV 254 nm.

### EXAMPLE 28

### N-(1-Methyl-3-phenyl-propyl)-3-trifluoromethyl-benzenesulfonamide

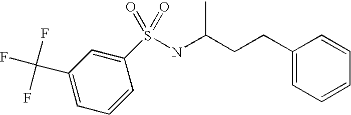

¹H NMR (299.944 MHz, CDCl₃) δ 8.16 (s, 1H), 8.05 (d, J=7.9 Hz, 1H), 7.84 (d, J=7.9 Hz, 1H), 7.66 (t, J=7.9 Hz, 1H), 7.29-7.16 (m, 3H), 7.07-7.04 (m, 2H), 4.50 (d, J=8.6 Hz, 1H), 3.42 (dq, J=8.3, 6.6 Hz, 1H), 2.57 (ddd, J=30.5, 14.1, 8.0 Hz, 2H), 1.73 (td, J=7.8, 6.7 Hz, 2H), 1.11 (d, J=6.6 Hz, 3H)

LC (method A) rt=6.2 min. UV 254 nm.

### EXAMPLE 29

### Biphenyl-4-sulfonic acid (1-methyl-3-phenyl-propyl)-amide

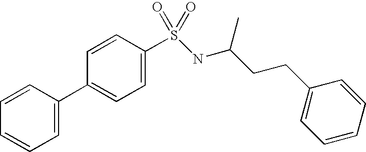

APCI-MS m/z: 366.2 [MH+]. LC (method A) rt=6.5 min. UV 254 nm.

### EXAMPLE 30

### 5-Bromo-thiophene-2-sulfonic acid (1-methyl-3-phenyl-propyl)-amide

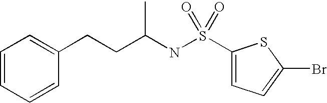

¹H NMR (299.944 MHz, CDCl₃) δ 7.29-7.20 (m, 3H), 7.19-7.12 (m, 1H), 7.09-7.04 (m, 2H), 7.00 (d, J=4.0 Hz, 1H), 4.50 (d, J=8.1 Hz, 1H), 3.40 (quintet, J=6.8 Hz, 1H), 2.58 (td, J=7.9, 5.3 Hz, 2H), 1.72 (dd, J=20.2, 2.2 Hz, 2H), 1.13 (d, J=6.6 Hz, 3H)

LC (method A) rt=6.1 min. UV 254 nm.

### EXAMPLE 31

### 4-n-Butoxy-N-(1-methyl-3-phenyl-propyl)-benzenesulfonamide

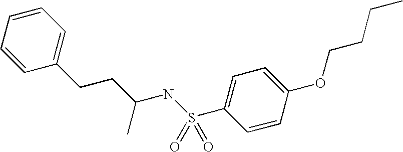

APCI-MS m/z: 362.2 [MH+].

LC (method A) rt=6.7 min. UV 254 nm.

### EXAMPLE 32

### 2,4,6-Trimethyl-N-(1-methyl-3-phenyl-propyl)-benzenesulfonamide

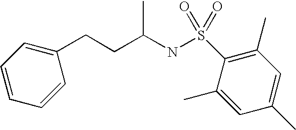

¹H NMR (299.944 MHz, CDCl₃) δ 7.31-7.16 (m, 3H), 7.05-7.00 (m, 4H), 4.43 (s, 1H), 3.33 (t, J=6.5 Hz, 1H), 2.67 (s, 6H), 2.64-2.47 (m, 2H), 2.36 (s, 3H), 1.75-1.67 (m, 2H), 1.14 (d, J=6.6 Hz, 3H)

APCI-MS m/z: 332.2 [MH+].

LC (method A) rt=6.4 min. UV 254 nm.

### EXAMPLE 33

### N-(1-Methyl-3-phenyl-propyl)-3-p-tolyloxy-benzenesulfonamide

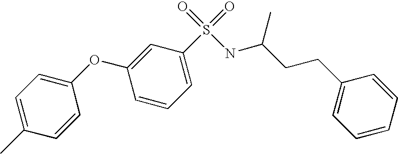

¹H NMR (299.944 MHz, CDCl₃) δ 7.57-7.53 (m, 1H), 7.29-7.14 (m, 6H), 7.08-7.04 (m, 2H), 6.91 (dt, J=8.9, 2.4 Hz, 2H), 7.46-7.41 (m, 2H), 4.57 (s, 1H), 3.38 (q, J=6.5 Hz, 1H), 2.65-2.46 (m, 2H), 2.36 (s, 3H), 1.69 (td, J=8.0, 6.6 Hz, 2H), 1.09 (d, J=6.6 Hz, 3H)

APCI-MS m/z: 396.2 [MH+].

LC (method A) rt=6.9 min. UV 254 nm.

### EXAMPLE 34

### N-[2-(2,6-Dimethyl-phenoxy)-1-methyl-ethyl]-3-nitro-benzenesulfonamide

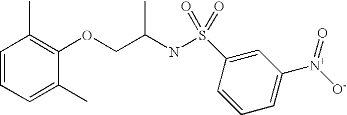

LC (method A) rt=5.9 min. UV 254 nm.

### EXAMPLE 35

### 4-Bromo-N-[2-(2,6-dimethyl-phenoxy)-1-methyl-ethyl]-benzenesulfonamide

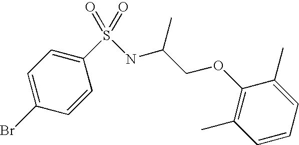

LC (method A) rt=6.4 min. UV 254 nm.

### EXAMPLE 36

### N-{4-[2-(2,6-Dimethyl-phenoxy)-1-methyl-ethylsulfamoyl]-phenyl}-acetamide

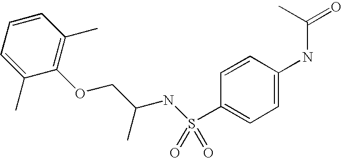

APCI-MS m/z: 377.2 [MH+].

LC (method A) rt=5.0 min. UV 254 nm.

### EXAMPLE 37

### N-[2-(2,6-Dimethyl-phenoxy)-1-methyl-ethyl]-4-nitro-benzenesulfonamide

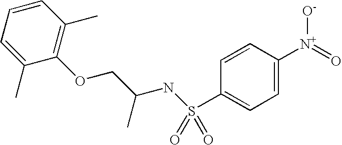

LC (method A) rt=6.0 min. UV 254 nm.

### EXAMPLE 38

### 4-Bromo-N-[2-(2,6-dimethyl-phenoxy)-1-methyl-ethyl]-2-methyl-benzenesulfonamide

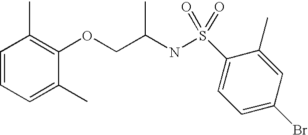

APCI-MS m/z: 412.1, 414.1 [MH+].

LC (method A) rt=6.7 min. UV 254 nm.

### EXAMPLE 39

### N-[2-(2,6-Dimethyl-phenoxy)-1-methyl-ethyl]-4-methoxy-benzenesulfonamide

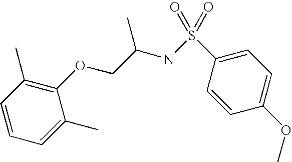

APCI-MS m/z: 350.2 [MH+].

LC (method A) rt=5.8 min. UV 254 nm.

### EXAMPLE 40

### N-[2-(2,6-Dimethyl-phenoxy)-1-methyl-ethyl]-4-trifluoromethoxy-benzenesulfonamide

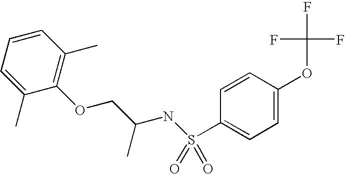

LC (method A) rt=6.6 min. UV 254 nm.

### EXAMPLE 41

### 4-tert-Butyl-N-[2-(2,6-dimethyl-phenoxy)-1-methyl-ethyl]-benzenesulfonamide

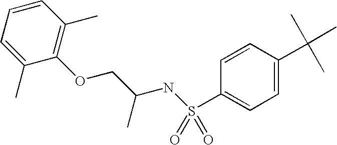

APCI-MS m/z: 376.3 [MH+].

LC (method A) rt=6.9 min. UV 254 nm.

### EXAMPLE 42

### 4-Cyano-N-[2-(2,6-dimethyl-phenoxy)-1-methyl-ethyl]-benzenesulfonamide

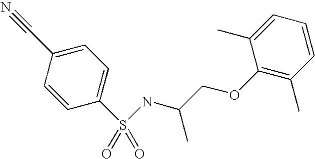

LC (method A) rt=5.7 min. UV 254 nm.

### EXAMPLE 43

### N-[2-(2,6-Dimethyl-phenoxy)-1-methyl-ethyl]-4-phenoxy-benzenesulfonamide

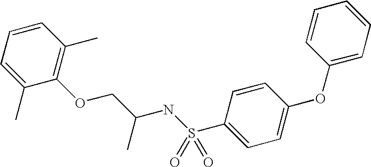

APCI-MS m/z: 412.3 [MH+].

LC (method A) rt=6.8 min. UV 254 nm.

### EXAMPLE 44

### 4′-Fluoro-biphenyl-4-sulfonic acid [2-(2,6-dimethyl-phenoxy)-1-methyl-ethyl]-amide

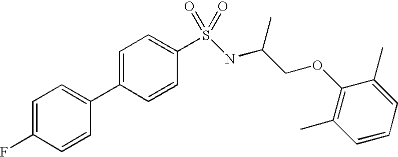

APCI-MS m/z: 414.2 [MH+].

LC (method A) rt=6.8 min. UV 254 nm.

### EXAMPLE 45

### N-[2-(2,6-Dimethyl-phenoxy)-1-methyl-ethyl]-4-propyl-benzenesulfonamide

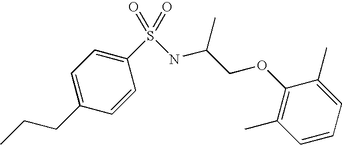

APCI-MS m/z: 362.2 [MH+].

LC (method A) rt=6.8 min. UV 254 nm.

### EXAMPLE 46

### N-[2-(2,6-Dimethyl-phenoxy)-1-methyl-ethyl]-4-(4-fluoro-phenoxy)-benzenesulfonamide

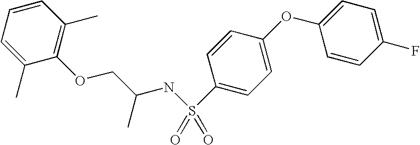

APCI-MS m/z: 430.1 [MH+].

LC (method A) rt=6.8 min. UV 254 nm.

### EXAMPLE 47

### N-[2-(2,6-Dimethyl-phenoxy)-1-methyl-ethyl]-4-(1,1-dimethyl-propyl)-benzenesulfonamide

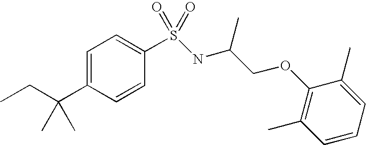

APCI-MS m/z: 390.2 [MH+].

LC (method A) rt=7.4 min. UV 254 nm.

### EXAMPLE 48

### Naphthalene-2-sulfonic acid [2-(2,6-dimethyl-phenoxy)-1-methyl-ethyl]-amide

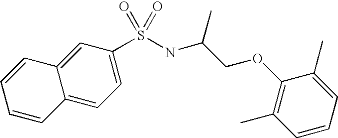

APCI-MS m/z: 370.1 [MH+].

LC (method A) rt=6.4 min. UV 254 nm.

### EXAMPLE 49

### Biphenyl-4-sulfonic acid [2-(2,6-dimethyl-phenoxy)-1-methyl-ethyl]-amide

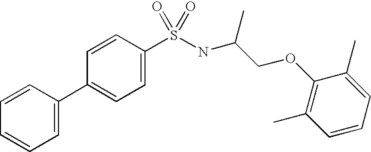

APCI-MS m/z: 396.2 [MH+].

LC (method A) rt=6.8 min. UV 254 nm.

### EXAMPLE 50

### 5-Bromo-thiophene-2-sulfonic acid [2-(2,6-dimethyl-phenoxy)-1-methyl-ethyl]-amide

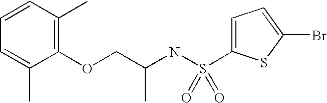

LC (method A) rt=6.4 min. UV 254 nm.

### EXAMPLE 51

### 2-Bromo-N-[2-(2,6-dimethyl-phenoxy)-1-methyl-ethyl]-benzenesulfonamide

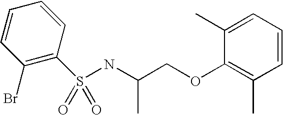

APCI-MS m/z: 398.0, 400.0 [MH+].

LC (method A) rt=6.2 min. UV 254 nm.

### EXAMPLE 52

### N-[2-(2,6-Dimethyl-phenoxy)-1-methyl-ethyl]-3-methoxy-benzenesulfonamide

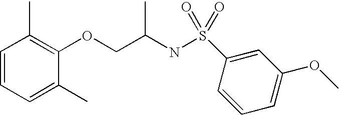

APCI-MS m/z: 350.2 [MH+].

LC (method A) rt=6.0 min. UV 254 nm.

### EXAMPLE 53

### 4-n-Butoxy-N-[2-(2,6-dimethyl-phenoxy)-1-methyl-ethyl]-benzenesulfonamide

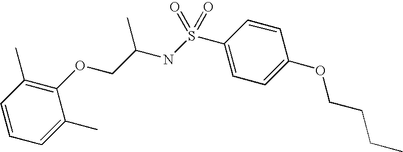

APCI-MS m/z: 392.2 [MH+].

LC (method A) rt=7.0 min. UV 254 nm.

### EXAMPLE 54

### N-[2-(2,6-Dimethyl-phenoxy)-1-methyl-ethyl]-4-(pyridin-2-yloxy)-benzenesulfonamide

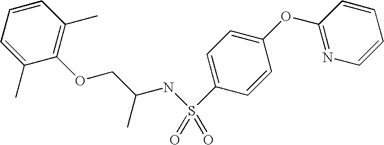

APCI-MS m/z: 413.2 [MH+].

LC (method A) rt=6.0 min. UV 254 nm.

### EXAMPLE 55

### N-[2-(2,6-Dimethyl-phenoxy)-1-methyl-ethyl]-2,4,6-trimethyl-benzenesulfonamide

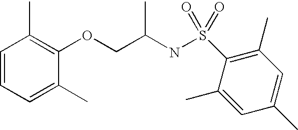

APCI-MS m/z: 362.2 [MH+].

LC (method A) rt=6.8 min. UV 254 nm.

### EXAMPLE 56

### N-[2-(2,6-Dimethyl-phenoxy)-1-methyl-ethyl]-3-p-tolyloxy-benzenesulfonamide

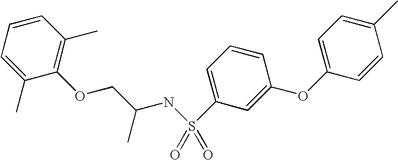

APCI-MS m/z: 426.2 [MH+].

LC (method A) rt=7.1 min. UV 254 nm.

### EXAMPLE 57

### 4-Bromo-2-methyl-N-(2-phenoxy-ethyl)-benzenesulfonamide

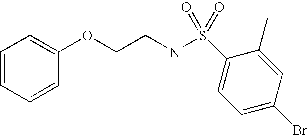

LC (method A) rt=5.9 min. UV 254 nm.

### EXAMPLE 58

### N-(2-Phenoxy-ethyl)-4-trifluoromethoxy-benzenesulfonamide

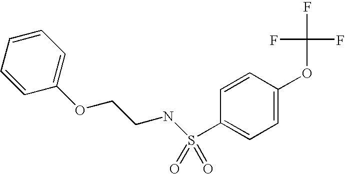

LC (method A) rt=5.9 min. UV 254 nm.

### EXAMPLE 59

### 4-(1,1-Dimethyl-propyl)-N-(2-phenoxy-ethyl)-benzenesulfonamide

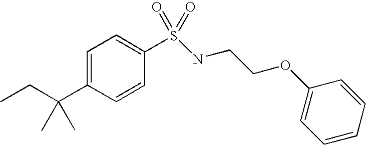

APCI-MS m/z: 348.2 [MH+].

LC (method A) rt=6.7 min. UV 254 nm.

### EXAMPLE 60

### Biphenyl-4-sulfonic acid (2-phenoxy-ethyl)-amide

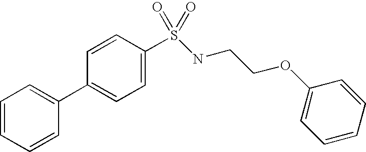

APCI-MS m/z: 354.1 [MH+].

LC (method A) rt=6.0 min. UV 254 nm.

### EXAMPLE 61

### 2,4,6-Trimethyl-N-(2-phenoxy-ethyl)-benzenesulfonamide

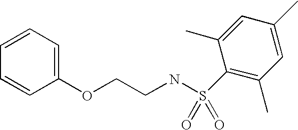

APCI-MS m/z: 320.2 [MH+].

LC (method A) rt=6.0 min. UV 254 nm.

### EXAMPLE 62

### 4-Bromo-N-(3-phenyl-propyl)-benzenesulfonamide

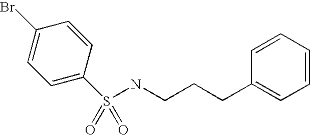

LC (method A) rt=6.0 min. UV 254 nm.

### EXAMPLE 63

### 4-Bromo-2-methyl-N-(3-phenyl-propyl)-benzenesulfonamide

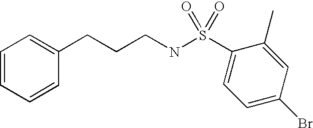

LC (method A) rt=6.3 min. UV 254 nm.

### EXAMPLE 64

### N-(3-Phenyl-propyl)-4-trifluoromethoxy-benzenesulfonamide

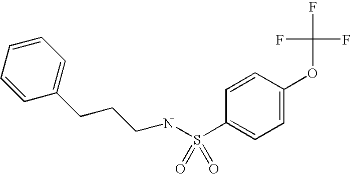

LC (method A) rt=6.2 min. UV 254 nm.

### EXAMPLE 65

### 4-Methoxy-2,3,6-trimethyl-N-(3-phenyl-propyl)-benzenesulfonamide

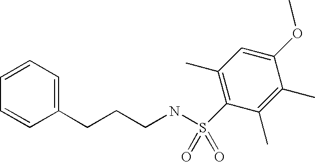

APCI-MS m/z: 348.2 [MH+].

LC (method A) rt=6.3 min. UV 254 nm.

### EXAMPLE 66

### 4-tert-Butyl-N-(3-phenyl-propyl)-benzenesulfonamide

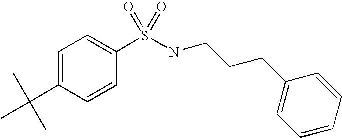

APCI-MS m/z: 332.2 [MH+].

LC (method A) rt=6.5 min. UV 254 nm.

### EXAMPLE 67

### 4-Phenoxy-N-(3-phenyl-propyl)-benzenesulfonamide

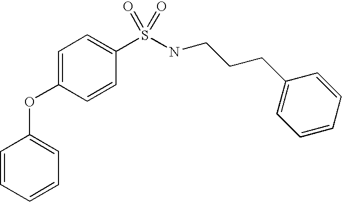

APCI-MS m/z: 368.2 [MH+].

LC (method A) rt=6.4 min. UV 254 nm.

### EXAMPLE 68

### 4′-Fluoro-biphenyl-4-sulfonic acid (3-phenyl-propyl)-amide

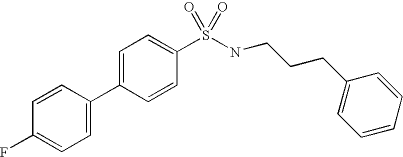

APCI-MS m/z: 370.1 [MH+].

LC (method A) rt=6.4 min. UV 254 nm.

### EXAMPLE 69

### N-(3-Phenyl-propyl)-4-propyl-benzenesulfonamide

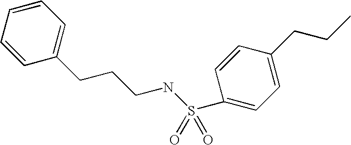

APCI-MS m/z: 318.2 [MH+].

LC (method A) rt=6.4 min. UV 254 nm.

### EXAMPLE 70

### 4-(4-Fluoro-phenoxy)-N-(3-phenyl-propyl)-benzenesulfonamide

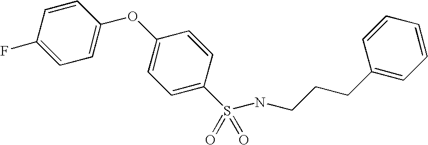

APCI-MS m/z: 386.2 [MH+].

LC (method A) rt=6.5 min. UV 254 nm.

### EXAMPLE 71

### 4-(1,1-Dimethyl-propyl)-N-(3-phenyl-propyl)-benzenesulfonamide

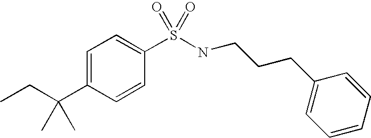

APCI-MS m/z: 346.3 [MH+].

LC (method A) rt=7.0 min. UV 254 nm.

### EXAMPLE 72

### Naphthalene-2-sulfonic acid (3-phenyl-propyl)-amide

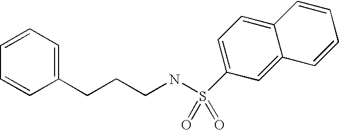

APCI-MS m/z: 326.2 [MH+].

LC (method A) rt=6.0 min. UV 254 nm.

### EXAMPLE 73

### Biphenyl-4-sulfonic acid (3-phenyl-propyl)-amide

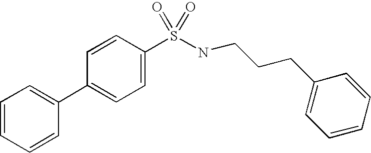

APCI-MS m/z: 352.1 [MH+].

LC (method A) rt=6.4 min. UV 254 nm.

### EXAMPLE 74

### 5-Bromo-thiophene-2-sulfonic acid (3-phenyl-propyl)-amide

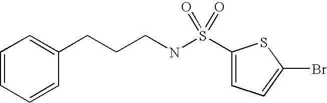

LC (method A) rt=6.0 min. UV 254 nm.

### EXAMPLE 75

### 2,4,6-Trimethyl-N-(3-phenyl-propyl)-benzenesulfonamide

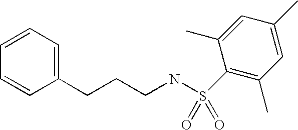

APCI-MS m/z: 318.2 [MH+].

LC (method A) rt=6.0 min. UV 254 nm.

### EXAMPLE 76

### N-(3-Phenyl-propyl)-3-p-tolyloxy-benzenesulfonamide

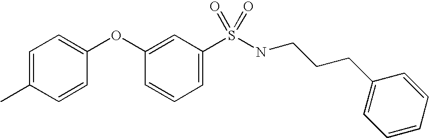

APCI-MS m/z: 382.1 [MH+].

LC (method A) rt=6.7 min. UV 254 nm.

### EXAMPLE 77

### N-[(1S)-2-(5-Isoquinolinyloxy)-1-methylethyl]-2,4,6-trimethylbenzenesulfonamide

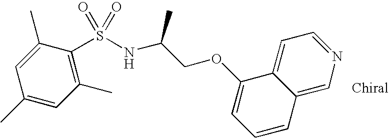

### Step 1: (2S)-2-[(Mesitylsulfonyl)amino]propyl 2,4,6-trimethylbenzenesulfonate

L-Alaninol (4.8 g, 64 mmole) and 2-mesitylenesulfonyl chloride (30 g, 137 mmole) were dissolved in 200 mL pyridine and stirred at room temperature overnight. The mixture was evaporated, dissolved in ethyl acetate (200 ml) and washed with 1M HCl/aq, sat. NaHCO₃/aq. The organic layer was dried, concentrated and purified on a silica gel column chromatography (heptane-ethylacetate).

APCI-MS m/z: 440.1 [MH+].

### Step 2: N-[(1S)-2-(5-Isoquinolinyloxy)-1-methylethyl]-2,4,6-trimethylbenzenesulfonamide

(2S)-2-[(Mesitylsulfonyl)amino]propyl 2,4,6-trimethylbenzenesulfonate (263 mg, 0.6 mmole) was added to a slurry containing Cs₂CO₃ (487 mg, 1.5 mmole) and 5-Hydroxyisoquinoline (145 mg, 1 mmole) in 2.5 mL DMF. The reaction mixture was stirred overnight in room temperature before it was diluted with ethyl acetate (20 mL) and washed with 1MHCl/aq. The organic layer was dried, concentrated and purified on HPLC-Qs.

¹H NMR (299.946 MHz, DMSO) δ 9.54 (s, 1H), 8.54 (d, J=6.2 Hz, 1H), 8.11 (d, J=6.2 Hz, 1H), 7.84 (dd, J=15.7, 8.5 Hz, 2H), 7.67 (t, J=8.1 Hz, 1H), 7.23 (d, J=7.3 Hz, 1H), 6.83 (d, J=0.4 Hz, 2H), 4.04-3.92 (m, 2H), 3.65 (dq, J=13.2, 6.6 Hz, 1H), 2.50 (s, 6H), 2.11 (d, J=11.6 Hz, 3H), 1.16 (d, J=6.8 Hz, 3H)

APCI-MS m/z: 385.1 [MH+].

Examples 78-83 were synthesised by a method analogous to that described in Example 77 using (2S)-2-[(mesitylsulfonyl)amino]propyl 2,4,6-trimethylbenzenesulfonate and the corresponding starting materials.

### EXAMPLE 78

### N-[(1S)-2-(1H-Indol-4-yloxy)-1-methylethyl]-2,4,6-trimethylbenzenesulfonamide

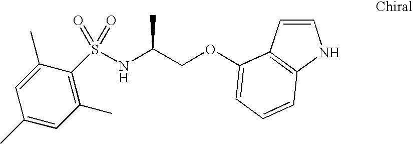

¹H NMR (299.946 MHz, DMSO) δ 10.94 (s, 1H), 7.66 (d, J=8.6 Hz, 1H), 7.10 (t, J=2.8 Hz, 1H), 6.93-6.80 (m, 4H), 6.23-6.16 (m, 2H), 3.85 (dd, J=9.7, 5.7 Hz, 1H), 3.69 (dd, J=9.7, 6.6 Hz, 2H), 3.46-3.37 (m, 1H), 2.50 (s, 6H), 2.17 (s, 3H), 1.03 (d, J=6.8 Hz, 2H)

APCI-MS m/z: 373.1 [MH+].

### EXAMPLE 79

### 2,4,6-Trimethyl-N-[(1S)-1-methyl-2-(5-quinolinyloxy)ethyl]benzenesulfonamide

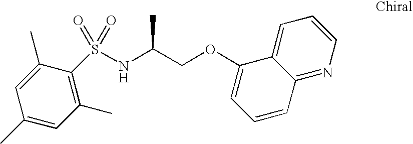

¹H NMR (299.946 MHz, DMSO) δ 9.13 (dd, J=4.8, 1.7 Hz, 1H), 8.79 (dd, J=8.4, 0.7 Hz, 1H), 7.88 (d, J=8.6 Hz, 1H), 7.65 (d, J=8.6 Hz, 1H), 7.83-7.75 (m, 2H), 7.04 (d, J=7.7 Hz, 1H), 6.82 (s, 2H), 6.72 (s, 1H), 4.06-3.94 (m, 2H), 3.70-3.62 (m, 1H), 2.50 (s, 6H), 2.13 (s, 3H), 1.17 (d, J=6.8 Hz, 2H)

APCI-MS m/z: 385.3 [MH+].

### EXAMPLE 80

### N-[(1S)-2-(1,3-Benzodioxol-5-yloxy)-1-methylethyl]-2,4,6-trimethylbenzenesulfonamide

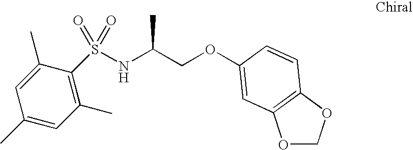

¹H NMR (299.946 MHz, DMSO) δ 7.62 (d, J=8.6 Hz, 1H), 6.95 (s, 2H), 6.68 (d, J=8.4 Hz, 1H), 6.23 (d, J=2.4 Hz, 1H), 6.08 (dd, J=8.5, 2.5 Hz, 1H), 5.89 (s, 2H), 3.67-3.53 (m, 2H), 3.39-3.30 (m, 1H), 2.50 (s, 6H), 2.21 (s, 3H), 1.00 (d, J=6.8 Hz, 3H)

APCI-MS m/z: 378.2 [MH+].

### EXAMPLE 81

### 2,4,6-Trimethyl-N-[(1S)-1-methyl-2-(4-quinolinyloxy)ethyl]benzenesulfonamide

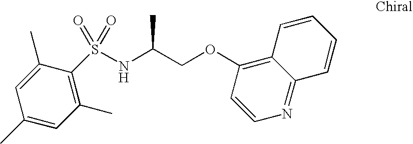

¹H NMR (299.946 MHz, DMSO) δ 8.10 (dd, J=8.1, 1.1 Hz, 1H), 7.90 (d, J=7.5 Hz, 1H), 7.81 (d, J=9.5 Hz, 1H), 7.74-7.64 (m, 2H), 7.42 (ddd, J=8.0, 6.3, 1.7 Hz, 1H), 6.56 (s, 2H), 6.15 (d, J=7.5 Hz, 1H), 4.40 (dd, J=14.6, 4.1 Hz, 1H), 3.91 (dd, J=14.7, 10.5 Hz, 1H), 3.62 (dd, J=6.2, 3.7 Hz, 1H), 2.20 (s, 6H), 2.13 (s, 3H), 1.21 (d, J=6.6 Hz, 3H)

APCI-MS m/z: 385.1 [MH+].

### EXAMPLE 82

### 2,4,6-Trimethyl-N-[(1S)-1-methyl-2-(4-quinazolinyloxy)ethyl]benzenesulfonamide

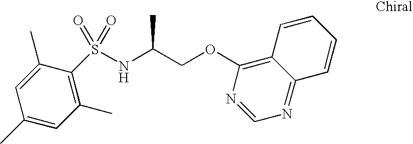

¹H NMR (299.946 MHz, DMSO) δ 8.08 (s, 1H), 7.96 (dd, J=7.9, 1.1 Hz, 1H), 7.82-7.76 (m, 1H), 7.73 (d, J=9.4 Hz, 1H), 7.57 (dd, J=8.0, 0.3 Hz, 1H), 7.49 (ddd, J=8.1, 7.1, 1.1 Hz, 1H), 6.52 (s, 2H), 3.98 (dd, J=12.1, 2.8 Hz, 1H), 3.70-3.53 (m, 2H), 2.36 (s, 6H), 1.91 (s, 3H), 1.13 (d, J=6.4 Hz, 3H)

APCI-MS m/z: 386.2 [MH+].

### EXAMPLE 83

### 2,4,6-Trimethyl-N-[(1S)-1-methyl-2-(8-quinolinyloxy)ethyl]benzenesulfonamide

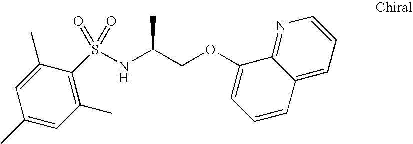

¹H NMR (299.946 MHz, DMSO) δ 9.14 (dd, J=5.0, 1.5 Hz, 1H), 9.02 (d, J=8.1 Hz, 1H), 8.04 (dd, J=8.3, 5.0 Hz, 1H), 7.82 (d, J=8.1 Hz, 1H), 7.73 (t, J=8.1 Hz, 1H), 7.41 (d, J=7.3 Hz, 1H), 6.76 (dd, J=0.3, 4.1 Hz, 2H), 4.21 (dd, J=10.3, 5.3 Hz, 2H), 4.04 (dd, J=10.3, 5.9 Hz, 1H), 3.70 (dd, J=20.9, 5.7 Hz, 1H), 2.11 (d, J==7.0 Hz, 3H), 1.24 (d, J=6.8 Hz, 3H), 2.50 (s, 6H)

APCI-MS m/z: 385.1 [MH+].

### EXAMPLE 84

### 5-Fluoro-2-({(2S)-2-[mesitylsulfonyl)amino]propyl}oxy)benzamide

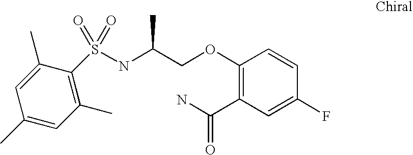

### (2S)-2-[(Mesitylsulfonyl)amino]propyl 2,4,6-trimethylbenzenesulfonate

L-Alaninol (4.8 g, 64 mmole) and 2-mesitylenesulfonyl chloride (30 g, 137 mmole) were dissolved in 200 mL pyridine and stirred at room temperature overnight. The mixture was evaporated, dissolved in ethyl acetate (200 ml) and washed with 1M HCl/aq, sat. NaHCO₃/aq. The organic layer was dried, concentrated and purified on a silica gel column chromatography (heptane-ethyl acetate).

APCI-MS m/z: 440.1 [MH+].

### Methyl 5-fluoro-2-hydroxybenzoate

5-Fluoro-2-hydroxybenzoic acid (468 mg, 3 mmole) was refluxed in methanol (20 mL+6 drops of cone H₂SO₄) overnight followed by evaporation to dryness. The product was used in next step without further purification.

### 5-Fluoro-2-hydroxybenzamide

Methyl 5-fluoro-2-hydroxybenzoate was dissolved in 37% NH₃/aq (20 mL) and stirred at 50° C. for 60 hours. The solution was concentrated, diluted with ethylacetate (20 mL) and washed with brine. The product was used in the next step without any further purification.

APCI-MS m/z: 156.0 [MH+].

### Aryl Ether Formation:

### 5-Fluoro-2-({(2S)-2-[(mesitylsulfonyl)amino]propyl}oxy)benzamide

(2S)-2-[(Mesitylsulfonyl)amino]propyl 2,4,6-trimethylbenzenesulfonate (263 mg, 0.6 mmole) was added to a slurry containing Cs₂CO₃ (487 mg, 1.5 mmole) and 5-fluoro-2-hydroxybenzamide (app. 1 mmole) in 2.5 mL DMF. The reaction mixture was stirred overnight in room temperature before it was diluted with ethylacetate (20 mL) and washed with 1M HCl/aq. The organic layer was dried, concentrated and purified on HPLC-C₁₈.

¹H NMR (299.946 MHz, DMSO) δ 7.79 (d, 8.4 Hz, 1H), 7.63 (s, 2H), 7.50 (dd, J=9.5, 3.3 Hz, 1H), 7.20 (ddd, J=9.1, 7.7, 3.4 Hz, 1H), 6.99-6.88 (m, 3H), 3.87 (d, J=5.9 Hz, 2H), 3.56-3.45 (m, 1H), 2.50 (s, 6H), 2.18 (s, 3H), 0.93 (d, J=6.8 Hz, 3H)

APCI-MS m/z: 395.2 [MH+].

Examples 85-95 were synthesised by a method analogous to that described in Example 84 using the corresponding starting materials.

### EXAMPLE 85

### 2-({(2S)-2-[(Mesitylsulfonyl)amino]propyl}oxy)-5-methylbenzamide

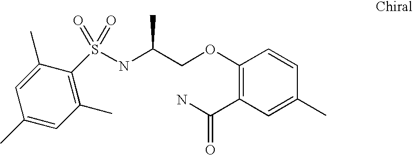

¹H NMR (299.946 MHz, DMSO) δ 7.78 (d, J=8.6 Hz, 1H), 7.59-7.51 (m, 2H), 7.40 (s, 1H), 7.14 (mult, 1H), 6.92 (s, 2H), 6.78 (d, J=8.4 Hz, 1H), 3.83 (d, J=5.8 Hz, 2H), 3.50 (dd, J=8.3, 6.6 Hz, 1H), 2.50 (s, 6H), 2.20 (s, 3H), 2.18 (d, J=3.1 Hz, 3H), 0.91 (d, J=6.8 Hz, 3H)

APCI-MS m/z: 391.1 [MH+].

### EXAMPLE 86

### 2-Hydroxy-6-({(2S)-2-[(mesitylsulfonyl)amino]propyl}oxy)benzamide

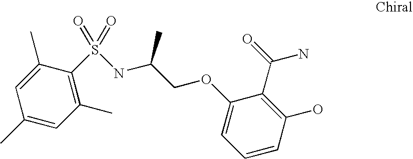

¹H NMR (299.946 MHz, DMSO) δ 8.07 (d, J=22.4 Hz, 2H), 7.79 (d, J=8.4 Hz, 1H), 7.20 (t, J=8.3 Hz, 1H), 6.92 (s, 2H), 6.39 (ddd, J=21.5, 8.3, 0.8 Hz, 2H), 3.96-3.79 (m, 2H), 3.66-3.52 (m, 1H), 2.50 (s, 6H), 2.19 (s, 3H), 0.88 (d, J=6.6 Hz, 3H)

APCI-MS m/z: 393.2 [MH+].

### EXAMPLE 87

### 5-Chloro-2-({(2S)-2-[(mesitylsulfonyl)amino]propyl}oxy)benzamide

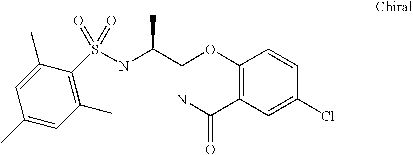

¹H NMR (299.946 MHz, DMSO) δ 7.79 (d, J=8.4 Hz, 1H), 7.71 (t, J=2.5 Hz, 1H), 7.66-7.60 (m, 2H), 7.39 (dd, J=8.8, 2.9 Hz, 1H), 6.97 (d, J=9.0 Hz, 1H), 6.90 (s, 2H), 3.90 (d, J=5.9 Hz, 2H), 3.53 (dd, J=20.7, 5.9 Hz, 1H), 2.50 (s, 6H), 2.18 (s, 3H), 0.94 (d, J=6.8 Hz, 3H)

APCI-MS m/z: 411.1 [MH+].

### EXAMPLE 88

### 2-({(2S)-2-[(Mesitylsulfonyl)amino]propyl}oxy)-4-methylbenzamide

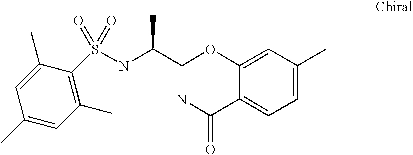

¹H NMR (299.946 MHz, DMSO) δ 7.80 (d, J=8.4 Hz, 1H), 7.69 (d, J=1.1 Hz, 1H), 7.51 (s, 1H), 7.35 (s, 1H), 6.91 (s, 2H), 6.77 (d, 7.9 Hz, 1H), 6.73 (s, 1H), 3.87 (d, J=5.7 Hz, 2H), 3.59-3.45 (m, 1H), 2.50 (s, 6H), 2.24 (s, 3H), 2.17 (s, 3H), 0.92 (d, J=6.8 Hz, 3H)

APCI-MS m/z: 391.1 [MH+].

### EXAMPLE 89

### 2-({(2S)-2-[(Mesitylsulfonyl)amino]propyl}oxy)benzamide

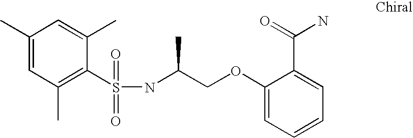

¹H NMR (399.988 MHz, CDCl₃) δ 8.05 (dd, J=7.8, 1.7 Hz, 1H), 7.92-7.82 (m, 1H), 7.37 (s, 1H), 7.00 (t, J=1.6 Hz, 2H), 6.94 (s, 2H), 6.80 (d, J=8.2 Hz, 1H), 5.73-5.60 (m, 1H), 4.05-3.94 (m, 2H), 3.89-3.78 (m, 1H), 2.66 (s, 6H), 2.29 (s, 3H), 1.13 (d, J=6.8 Hz, 3H)

APCI-MS m/z: 377.2 [MH+].

### EXAMPLE 90

### 4-Fluoro-2-({(2S)-2-[(mesitylsulfonyl)amino]propyl}oxy)benzamide

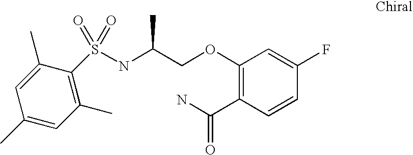

¹H NMR (299.946 MHz, DMSO) δ 7.87-7.79 (m, 2H), 7.49 (s, 2H), 6.94-6.72 (m, 4H), 3.92-3.87 (m, 2H), 3.54 (dd, J=8.2, 6.7 Hz, 1H), 2.50 (s, 6H), 2.17 (s, 3H), 0.93 (d, J=6.8 Hz, 3H)

APCI-MS m/z: 395.2 [MH+].

### EXAMPLE 91

### 4-Chloro-2-({(2S)-2-[(mesitylsulfonyl)amino]propyl}oxy)benzamide

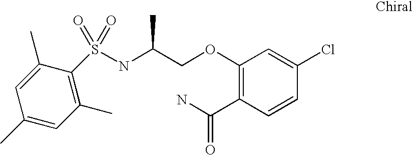

¹H NMR (299.946 MHz, DMSO) δ 7.80 (d, J=8.4 Hz, 2H), 7.76 (d, J=8.4 Hz, 2H), 7.55 (s, 2H), 7.53 (s, 2H), 7.06-6.99 (m, 2H), 6.90 (s, 2H), 3.91 (d, J=5.9 Hz, 2H), 3.57-3.48 (m, 10H), 2.50 (s, 10H), 2.18 (s, 3H), 0.94 (d, J=6.8 Hz, 3H)

APCI-MS m/z: 411.1 [MH+].

### EXAMPLE 92

### 5-Cyano-2-({(2S)-2-[(mesitylsulfonyl)amino]propyl}oxy)benzamide

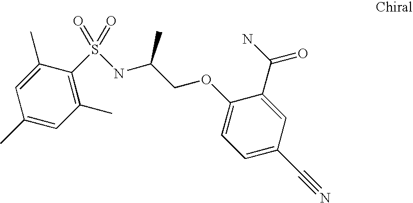

¹H NMR (299.944 MHz, CDCl₃) δ 8.27 (d, J=2.2 Hz, 1H), 7.95 (s, 1H), 7.69 (dd, J=8.6, 2.4 Hz, 1H), 6.97-6.91 (m, 3H), 6.85 (s, 1H), 6.04 (d, J=7.5 Hz, 1H), 4.15 (dd, J=9.2, 3.9 Hz, 1H), 4.06-3.86 (m, 2H), 2.67 (s, 6H), 2.31 (s, 3H), 1.05 (d, J=6.6 Hz, 3H)

APCI-MS m/z: 402.1 [MH+].

### EXAMPLE 93

### 2-({(2S)-2-[(Mesitylsulfonyl)amino]propyl}oxy)-5-methoxybenzamide

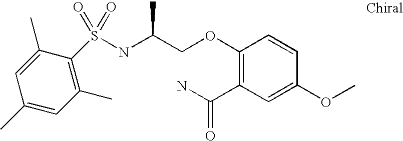

¹H NMR (299.946 MHz, DMSO) δ 7.78 (d, J=8.4 Hz, 1H), 7.61 (s, 1H), 7.49 (s, 1H), 7.32 (d, 3.1 Hz, 1H), 6.95-6.81 (m, 4H), 3.81 (d, J=5.7 Hz, 2H), 3.68 (s, 3H), 3.53-3.42 (m, 1H), 2.50 (s, 6H), 2.18 (s, 3H), 0.91 (d, J=6.8 Hz, 3H)

APCI-MS m/z: 407.2 [MH+].

### EXAMPLE 94

### 3-({(2S)-2-[(Mesitylsulfonyl)amino]propyl}oxy)-4-methylbenzamide

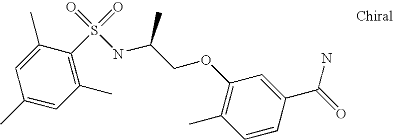

¹H NMR (299.946 MHz, DMSO) δ 7.84 (s, 1H), 7.67 (d, J==8.4 Hz, 1H), 7.31 (dd, J=1.6, 1.4 Hz, 1H), 7.23-7.17 (m, 2H), 7.10 (dd, J=1.1, 0.6 Hz, 1H), 6.92 (s, 2H), 3.75 (ddd, J=34.1, 9.7, 5.8 Hz, 2H), 3.51-3.41 (m, 1H), 2.50 (s, 6H), 2.16 (d, J=6.6 Hz, 3H), 2.01 (s, 3H), 1.04 (d, J=6.8 Hz, 3H)

APCI-MS m/z: 391.1 [MH+].

### EXAMPLE 95

### 2-({(2S)-2-[(Mesitylsulfonyl)amino]propyl}oxy)-4-methoxybenzamide

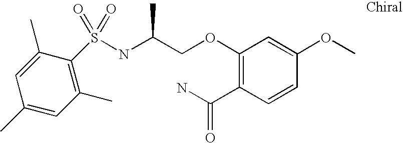

¹H NMR (299.946 MHz, DMSO) δ 7.84-7.76 (m, 2H), 7.44 (s, 1H), 7.26 (s, 1H), 6.91 (s, 2H), 6.54 (ddd, J=8.8, 4.0, 2.3 Hz, 1H), 6.41 (d, J=2.4 Hz, 1H), 3.91-3.86 (m, 2H), 3.74 (s, 3H), 3.54 (dd, J=8.2, 6.5 Hz, 1H), 2.50 (s, 6H), 2.17 (s, 3H), 0.91 (d, J=6.8 Hz, 3H)

APCI-MS m/z: 407.2 [MH+].

### EXAMPLE 96

### 2,5-Dichloro-N-[(1S)-2-(isoquinolin-5-yloxy)-1-methylethyl]thiophene-3-sulfonamide

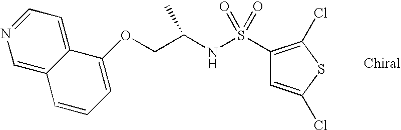

### 2-[(1S)-2-Hydroxy-1-methylethyl]-1H-isoindole-1,3(2H)-dione

Phthalic anhydride (50 mmole, 7.4 g) was dissolved in 100 mL toluene together with L-alaninol (50 mmole, 3.9 mL) and DIEA (5 mmole, 900 μL). The mixture was refluxed with continues removal of water with a Dean-Stark apparatus for two hours before it was washed with 1M HCl/aq, sat. NaHCO₃/aq. The organic layer was dried, concentrated and used in the next step without any further purification.

APCI-MS m/z: 206.0 [MH+].

### (2S)-2-(1,3-Dioxo-1,3-dihydro-2H-isoindol-2-yl)propyl 4-methylbenzenesulfonate

4-Methylbenzenesulfonyl chloride (43 mmole, 8.2 g) and 2-[(1S)-2-hydroxy-1-methylethyl]-1H-isoindole-1,3(2H)-dione (43 mmole, 8.8 g) were dissolved in pyridine (200 mL) and stirred overnight in room temperature. The mixture was evaporated, dissolved in ethyl acetate (200 ml) and washed with 1M HCl/aq, sat. NaHCO₃/aq. The organic layer was dried, concentrated and purified on a silica gel column chromatography (heptane-ethyl acetate).

APCI-MS m/z: 360.0 [MH+].

### 2-[(1S)-2-(Isoquinolin-5-yloxy)-1-methylethyl]-1H-isoindole-1,3(2H)-dione

(2S)-2-(1,3-Dioxo-1,3-dihydro-2H-isoindol-2-yl)propyl 4-methylbenzenesulfonate (8 mmole, 2.9 g) was added to a slurry containing Cs₂CO₃ (4 g, 12 mmole) and 5-hydroxyisoquinoline (1.3 g, 8.8 mmole) in 100 mL DMF. The reaction mixture was stirred for two hours at 100° C. before it was diluted with water (200 mL) and extracted with ethylacetate (3×150 mL). The combined organic layers were dried, concentrated and purified on a silica gel column chromatography (heptane-ethyl acetate).

### Amine Preparation

### [(1S)-2-(Isoquinolin-5-yloxy)-1-methylethyl]amine

2-[(1S)-2-(Isoquinolin-5-yloxy)-1-methylethyl]-1H-isoindole-1,3(2H)-dione (4.7 mmole, 1.56 g) was dissolved in ethanol (40 mL) together with hydrazine hydrate (14.1 mmole, 684 μL) and acetic acid (14.1 mmole, 805 μL) and refluxed for 3 hours. Solid material was removed by filtration and the solution was concentrated and purified on an ion exchange column (DOWEX 50WX2-400).

APCI-MS m/z: 203.1 [MH+].

### Sulfonamide Coupling:

### 2,5-Dichloro-N-[(1S)-2-(isoquinolin-5-yloxy)-1-methylethyl]thiophene-3-sulfonamide

2,5-Dichlorothiophene-3-sulfonyl chloride (100 μL, 0.3M/THF) was mixed with [(1S)-2-(isoquinolin-5-yloxy)-1-methylethyl]amine (100 μL, 0.3M/pyridine) and stirred overnight in ambient temperature before it was evaporated to dryness under reduced pressure. The residue was purified on HPLC-C₁₈.

APCI-MS m/z: 349.1 [MH+].

LC (method A) rt=3.2 min. UV 254 nm.

Examples 97-122 were synthesised by a method analogous to that described in Example 96 using the corresponding starting materials.

### EXAMPLE 97

### N-[(1S)-2-(Isoquinolin-5-yloxy)-1-methylethyl]-5-methyl-1-phenyl-1H-pyrazole-4-sulfonamide

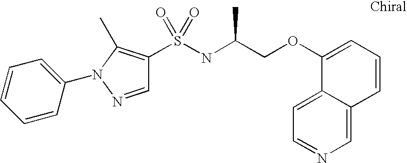

APCI-MS m/z: 423.2 [MH+].

LC (method A) rt=3.7 min. UV 254 nm.

### EXAMPLE 98

### 1-(Difluoromethyl)-N-[(1S)-2-(isoquinolin-5-yloxy)-1-methylethyl]-3,5-dimethyl-1H-pyrazole-4-sulfonamide

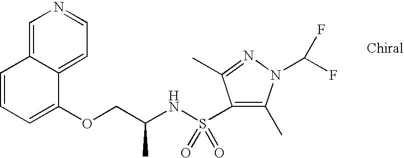

APCI-MS m/z: 411.1 [MH+].

LC (method A) rt=3.4 min. UV 254 nm.

### EXAMPLE 99

### N-[(1S)-2-(Isoquinolin-5-yloxy)-1-methylethyl]-2,5-dimethylfuran-3-sulfonamide

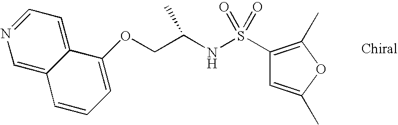

APCI-MS m/z: 361.1 [MH+].

LC (method A) rt=3.6 min. UV 254 nm.

### EXAMPLE 100

### 2,5-Dichloro-N-[(1S)-1-methyl-2-(quinolin-5-yloxy)ethyl]thiophene-3-sulfonamide

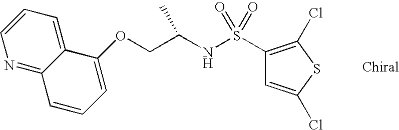

APCI-MS m/z: 416.9, 419.0 [MH+].

LC (method A) rt=4.0 min. UV 254 nm.

### EXAMPLE 101

### 3-Bromo-5-chloro-N-[(1S)-1-methyl-2-(quinolin-5-yloxy)ethyl]thiophene-2-sulfonamide

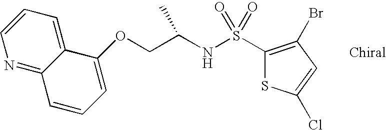

APCI-MS m/z: 460.9, 463.0 [MH+].

LC (method A) rt=4.1 min. UV 254 nm.

### EXAMPLE 102

### N-[(1S)-2-Isoquinolin-5-yloxy)-1-methylethyl]-5-[1-methyl-5-trifluoromethyl)-1H-pyrazol-3-yl]thiophene-2-sulfonamide

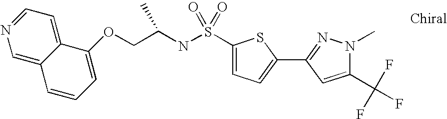

APCI-MS m/z: 497.0 [MH+].

LC (method A) rt=4.5 min. UV 254 nm.

### EXAMPLE 103

### 1-(Difluoromethyl)-N-[(1S)-2-(isoquinolin-5-yloxy)-1-methylethyl]-5-methyl-1H-pyrazole-4-sulfonamide

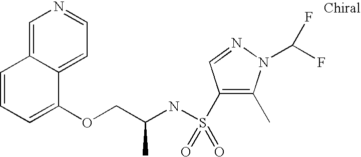

APCI-MS m/z: 397.1 [MH+].

LC (method A) rt=3.3 min. UV 254 nm.

### EXAMPLE 104

### 5-Methyl-N-[(1S)-1-methyl-2-(quinolin-5-yloxy)ethyl]-1-phenyl-1H-pyrazole-4-sulfonamide

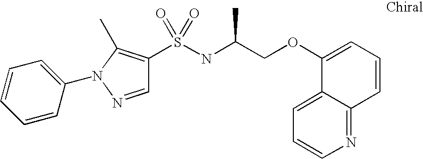

APCI-MS m/z: 416.1 [MH+].

LC (method A) rt=3.6 min. UV 254 nm.

### EXAMPLE 105

### 5-Chloro-N-[(1S)-2-(isoquinolin-5-yloxy)-1-methylethyl]thiophene-2-sulfonamide

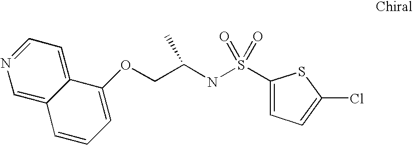

APCI-MS m/z: 383.0 [MH+].

LC (method A) rt=3.8 min. UV 254 nm.

### EXAMPLE 106

### 5-Chloro-N-[(1S)-1-methyl-2-(quinolin-5-yloxy)ethyl]thiophene-2-sulfonamide

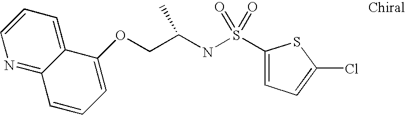

APCI-MS m/z: 383.0 [MH+].

LC (method A) rt=3.8 min. UV 254 nm.

### EXAMPLE 107

### Methyl 4-({[(1S)-2-(isoquinolin-5-yloxy)-1-methylethyl]-amino}sulfonyl)-2,5-dimethyl-3-furoate

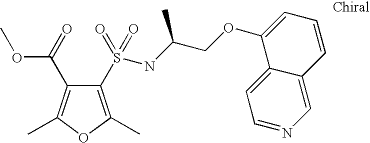

APCI-MS m/z: 419.2 [MH+].

LC (method A) rt=3.8 min. UV 254 nm.

### EXAMPLE 108

### N-[(1S)-2-(Isoquinolin-5-yloxy)-1-methylethyl]thiophene-3-sulfonamide

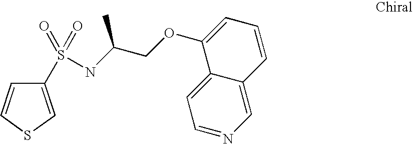

APCI-MS m/z: 349.1 [MH+].

LC (method A) rt=3.2 min. UV 254 nm.

### EXAMPLE 109

### 1-Ethyl-N-[(1S)-2-(isoquinolin-5-yloxy)-1-methylethyl]-1H-pyrazole-4-sulfonamide

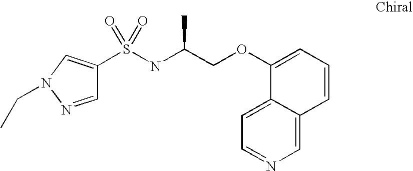

APCI-MS m/z: 361.1 [MH+].

LC (method A) rt=2.9 min. UV 254 nm.

### EXAMPLE 110

### 2-[((2S)-2-{[(2,5-Dichloro-3-thienyl)sulfonyl]amino}propyl)oxy]benzamide

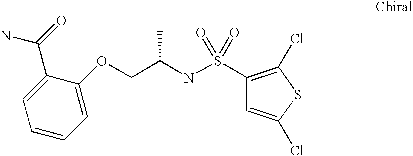

APCI-MS m/z: 409.0, 410.9 [MH+].

LC (method A) rt=4.7 min. UV 254 nm.

### EXAMPLE 111

### 1-(Difluoromethyl)-3,5-dimethyl-N-[(1S)-1-methyl-2-(quinolin-5-yloxy)ethyl]-1H-pyrazole-4-sulfonamide

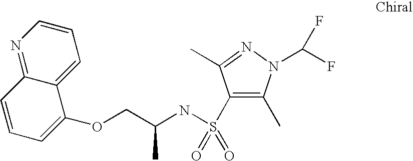

APCI-MS m/z: 411.1 [MH+].

LC (method A) rt=3.4 min. UV 254 nm.

### EXAMPLE 112

### N-[(1S)-1-Methyl-2-(quinolin-5-yloxy)ethyl]-5-[1-methyl-5-(trifluoromethyl)-1-pyrazol-3-yl]thiophene-2-sulfonamide

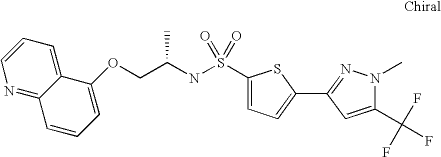

APCI-MS m/z: 497.0 [MH+].

LC (method A) rt=4.5 min. UV 254 nm.

### EXAMPLE 113

### 1-Ethyl-N-[(1S)-1-methyl-2-(quinolin-5-yloxy)ethyl]-1H-pyrazole-4-sulfonamide

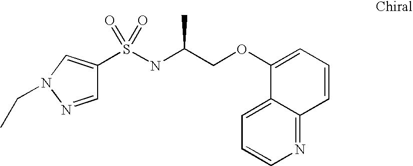

APCI-MS m/z: 361.1 [MH+].

LC (method A) rt=2.9 min. UV 254 nm.

### EXAMPLE 114

### 2-({(2S)-2-[({5-[1-Methyl-5-trifluoromethyl)-1H-pyrazol-3-yl]-2-thienyl}sulfonyl)-amino]propyl}oxy)benzamide

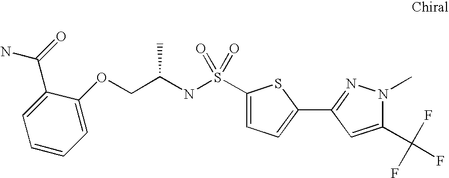

APCI-MS m/z: 489.1 [MH+].

LC (method A) rt=5.1 min. UV 254 nm.

### EXAMPLE 115

### 2-[((2S)-2-{[(2,5-Dimethyl-3-thienyl)sulfonyl]amino}propyl)oxy]benzamide

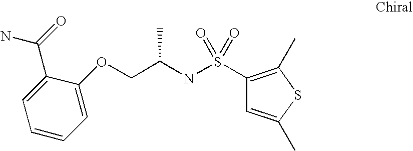

APCI-MS m/z: 369.1 [MH+].

LC (method A) rt=4.4 min. UV 254 nm.

### EXAMPLE 116

### 2,5-Dimethyl-N-[(1S)-1-methyl-2-(quinolin-5-yloxy)ethyl]furan-3-sulfonamide

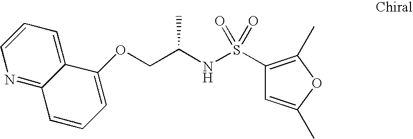

APCI-MS m/z: 361.1 [MH+].

LC (method A) rt=3.7 min. UV 254 nm.

### EXAMPLE 117

### 2-[((2S)-2-{[(2,5-Dimethyl-3-furyl)sulfonyl]amino}propyl)oxy]benzamide

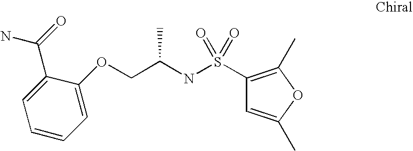

APCI-MS m/z: 353.2 [MH+].

LC (method A) rt=4.2 min. UV 254 nm.

### EXAMPLE 118

### 2-{[(2S)-2-({[1-(Difluoromethyl)-3,5-dimethyl-1H-pyrazol-4-yl]sulfonyl}amino)propyl]-oxy}benzamide

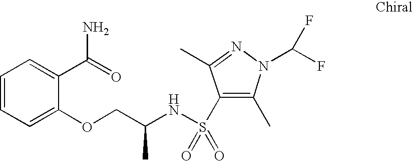

APCI-MS m/z: 403.0 [MH+].

LC (method A) rt=3.9 min. UV 254 nm.

### EXAMPLE 119

### 1-Ethyl-N-[(1S)-2-(isoquinolin-5-yloxy)-1-methylethyl]-3-methyl-1H-pyrazole-4-sulfonamide

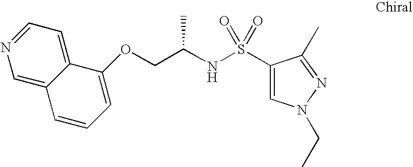

APCI-MS m/z: 375.2 [MH+].

LC (method A) rt=3.0 min. UV 254 nm.

### EXAMPLE 120

### N-[(1S)-2-(Isoquinolin-5-yloxy)-1-methylethyl]-1,3,5-trimethyl-1H-pyrazole-4-sulfonamide

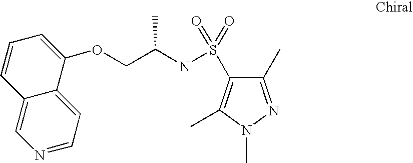

APCI-MS m/z: 375.1 [MH+].

LC (method A) rt=2.9 min. UV 254 nm.

### EXAMPLE 121

### N-[(1S)-2-(Isoquinolin-5-yloxy)-1-methylethyl]-3,5-dimethylisoxazole-4-sulfonamide

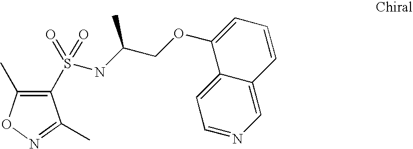

APCI-MS m/z: 362.2 [MH+].

LC (method A) rt=3.3 min. UV 254 nm.

### EXAMPLE 122

### N-[(1S)-2-(Isoquinolin-5-yloxy)-1-methylethyl]-2,5-dimethylthiophene-3-sulfonamide

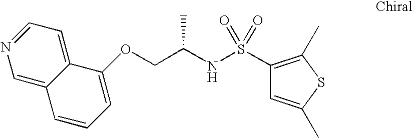

APCI-MS m/z: 377.2 [MH+].

LC (method A) rt=3.8 min. UV 254 nm.

### EXAMPLE 123

### 2,4,6-Trimethyl-N-{(1S)-1-methyl-2-[(8-methylquinolin-5-yl)amino]ethyl}-benzenesulfonamide

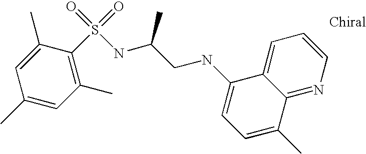

(2S)-2-[(Mesitylsulfonyl)amino]propyl 2,4,6-trimethylbenzenesulfonate was prepared as described in Example 77.

### 2,4,6-Trimethyl-N-{(1S)-1-methyl-2-[(8-methylquinolin-5-yl)amino]ethyl}benzenesulfonamide

(2S)-2-[(Mesitylsulfonyl)amino]propyl 2,4,6-trimethylbenzenesulfonate (132 mg, 0.3 mmole) and 8-methylquinolin-5-amine (47 mg, 0.3 mmole) were dissolved in NMP (1 mL) and heated to 130° C. for 2 hours. The reaction mixture was purified directly on HPLC-C₁₈.

¹H NMR (399.99 MHz, DMSO) δ 8.80 (d, J=5.2 Hz, 1H), 8.34 (d, J=9.4 Hz, 1H), 7.57 (d, J=8.4 Hz, 1H), 7.36 (dd, J=8.6, 4.1 Hz, 1H), 7.19 (d, J=7.8 Hz, 1H), 6.83 (s, 2H), 6.11 (d, J=7.8 Hz, 1H), 6.06 (t, J=5.6 Hz, 1H), 3.38 (q, J=7.1 Hz, 1H), 3.06 (dd, J=13.7, 8.1 Hz, 2H), 2.50 (s, 6H), 2.49 (s, 3H), 2.14 (s, 3H), 1.01 (d, J=6.6 Hz, 3H)

APCI-MS m/z: 398.1 [MH+].

Examples 124-129 were synthesised by a method analogous to that described in Example 123 using the corresponding starting materials.

### EXAMPLE 124

### 2,4,6-Trimethyl-N-{(1S)-1-methyl-2-[(6-methylquinolin-5-yl)amino]ethyl}-benzenesulfonamide

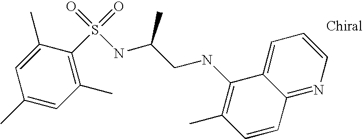

¹H NMR (399.99 MHz, DMSO) δ 8.80 (d, J=3.0 Hz, 1H), 8.34 (d, J=7.6 Hz, 1H), 7.57 (s, 1H), 7.36 (dd, J=8.4, 4.1 Hz, 1H), 7.19 (d, J=7.8 Hz, 1H), 6.83 (s, 2H), 6.11 (d, J=7.8 Hz, 1H), 6.07 (t, J=5.6 Hz, 1H), 3.40-3.33 (m, 1H), 3.06 (d, J=5.3 Hz, 2H), 2.50 (s, 6H), 2.50 s, 3H), 2.14 (s, 3H), 1.01 (d, J=6.5 Hz, 3H)

APCI-MS m/z: 398.1 [MH+].

### EXAMPLE 125

### N-[(1S)-2-(1H-Indazol-4-ylamino)-1-methylethyl]-2,4,6-trimethylbenzenesulfonamide

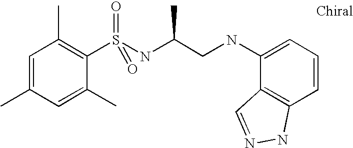

¹H NMR (399.991 MHz, cd3cn) δ 7.92 (s, 1H), 7.03 (d, J=7.7 Hz, 1H), 6.87 (t, J=7.8 Hz, 1H), 6.81 (s, 2H), 6.21 (d, J=7.4 Hz, 1H), 5.68 (d, J=8.1 Hz, 1H), 3.60-3.49 (m, 1H), 3.21 (mult, 2H), 2.51 (s, 6H), 2.18 (s, 3H), 1.14 (d, J=6.6 Hz, 3H)

APCI-MS m/z: 373.1 [MH+].

### EXAMPLE 126

### 2,4,6-Trimethyl-N-[(1S)-1-methyl-2-(quinolin-5-Ylamino)ethyl]benzenesulfonamide

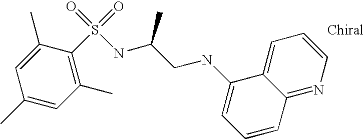

¹H NMR (299.946 MHz, cd3cn) δ 8.80 (d, J=4.0 Hz, 1H), 8.10 (d, J=8.6 Hz, 1H), 7.34 (mult, 3H), 6.74 (s, 2H), 6.36 (d, J=1.1 Hz, 1H), 5.68 (d, J=1.9 Hz, 1H), 5.23 (s, 1H), 3.57 (mult, 1H), 3.18 (mult, 2H), 2.51 (s, 6H), 2.12 (s, 3H), 1.17 (d, J=6.6 Hz, 3H)

APCI-MS m/z: 384.1 [MH+].

### EXAMPLE 127

### N-[(1S)-2-(1H-Indazol-6-ylamino)-1-methylethyl]-2,4,6-trimethylbenzenesulfonamide

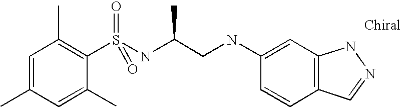

¹H NMR (399.991 MHz, cd3cn) δ 7.83 (s, 1H), 7.41 (d, J=8.7 Hz, 1H), 6.90 (s, 2H), 6.38 (dd, J=8.8, 1.9 Hz, 1H), 6.34 (s, 1H), 5.63 (d, J=8.1 Hz, 1H), 3.46 (t, J=6.5 Hz, 1H), 3.07 (td, J=13.4, 7.7 Hz, 2H), 2.56 (s, 6H), 1.10 (d, J=6.6 Hz, 3H), 2.17 (s, 3H)

APCI-MS m/z: 373.1 [MH+].

### EXAMPLE 128

### 2,4,6-Trimethyl-N-{(1S)-1-methyl-2-[(2-methylquinolin-5-yl)amino]ethyl}-benzenesulfonamide

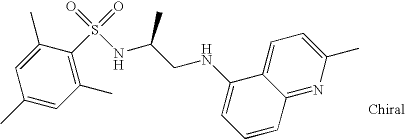

¹H NMR (399.991 MHz, cd3cn) δ 7.99 (d, J=8.7 Hz, 1H), 7.36 (t, J=8.0 Hz, 1H), 7.23 (d, J=8.7 Hz, 1H), 7.17 (d, J=8.4 Hz, 1H), 6.77 (s, 2H), 6.31 (d, J=1.1 Hz, 1H), 5.69 (d, J=6.7 Hz, 1H), 5.17 (s, 1H), 3.56 (d, J=6.0 Hz, 1H), 3.16 (mult, 2H), 2.64 (s, 3H), 2.52 (s, 6H), 2.14 (s, 3H), 1.17 (d, J=6.1 Hz, 3H)

APCI-MS m/z: 398.1 [MH+].

### EXAMPLE 129

### N-[(1S)-2-(1H-Indazol-5-ylamino)-1-methylethyl]-2,4,6-trimethylbenzenesulfonamide

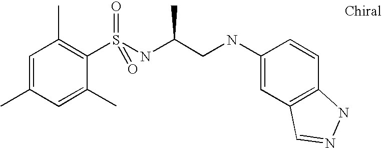

¹H NMR (399.991 MHz, cd3cn) δ 7.85 (s, 1H), 7.39 (d, J=8.6 Hz, 1H), 6.95 (s, 2H), 6.85 (s, 1H), 6.83 (d, J=2.1 Hz, 1H), 5.82 (d, J=8.2 Hz, 1H), 3.50 (t, J=6.4 Hz, 1H), 3.12 (mult, 1H), 2.57 (s, 6H), 2.21 (s, 3H), 1.06 (d, J=6.1 Hz, 3H)

APCI-MS m/z: 373.1 [MH+].

### EXAMPLE 130

### N-((1S)-2-{[2-Chloro-4-methylsulfonyl)phenyl]amino}-1-methylethyl)-2,4,6-dimethylbenzenesulfonamide

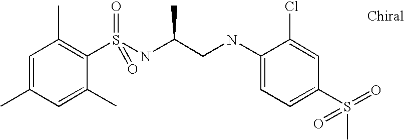

¹H NMR (399.99 MHz, DMSO) δ 7.63 (d, J=2.1 Hz, 1H), 7.55 (s, 1H), 7.47 (dd, J=8.7, 2.0 Hz, 1H), 6.89 (s, 2H), 6.58 (d, J=8.8 Hz, 1H), 6.16 (t, J=5.8 Hz, 1H), 3.22-3.03 (m, 6H), 0.51 (s, 6H), 2.20 (s, 3H), 1.01 (d, J=6.5 Hz, 3H)

APCI-MS m/z: 445.0 [MH+].

Examples 131-144 were prepared via the aryl ether formation as described in Example 4, using (2S)-2-[(mesitylsulfonyl)amino]propyl 2,4,6-trimethylbenzenesulfonate and the corresponding starting materials.

### EXAMPLE 131

### N-[(1S)-2-(4-Cyano-2,6-dimethylphenoxy)-1-methylethyl]-2,4,6-trimethylbenzenesulfonamide

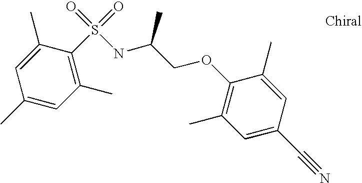

¹H NMR (299.946 MHz, DMSO) δ 7.76 (d, J=8.4 Hz, 1H), 7.50 (s, 2H), 7.01 (s, 2H), 3.82-3.71 (m, 0H), 3.57-3.37 (m, 3H), 2.55 (s, 6H), 2.24 (s, 3H), 2.10 (s, 6H), 1.13 (d, J=6.6 Hz, 3H)

APCI-MS m/z: 387.2 [MH+].

### EXAMPLE 132

### N-[(1S)-2-(3-Cyanophenoxy)-1-methylethyl]-2,4,6-trimethylbenzenesulfonamide

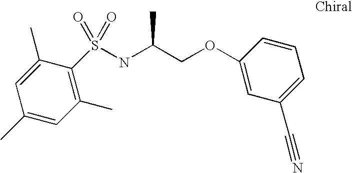

¹H NMR (299.946 MHz, DMSO) δ 7.72 (d, J=8.4 Hz, 1H), 7.44-7.30 (m, 2H), 7.03-6.98 (m, 2H), 6.95 (s, 2H), 3.82-3.77 (m, 2H), 2.52 (s, 6H), 2.24 (s, 3H), 1.09 (d, J=6.8 Hz, 3H)

APCI-MS m/z: 359.2 [MH+].

### EXAMPLE 133

### N-[(1S)-2-(3-Methoxyphenoxy)-1-methylethyl]-2,4,6-trimethylbenzenesulfonamide

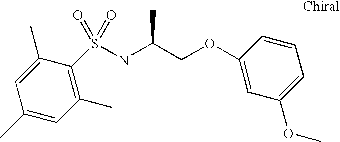

¹H NMR (299.946 MHz, DMSO) δ 7.68 (d, J=8.4 Hz, 1H), 7.11 (t, J=8.2 Hz, 1H), 7.00 (s, 2H), 6.47 (ddd, J=8.3, 2.4, 0.7 Hz, 1H), 6.28 (ddd, J=8.2, 2.3, 0.7 Hz, 1H), 6.21 (t, J=2.4 Hz, 1H), 3.79-3.63 (m, 2H), 3.48-3.36 (m, 1H), 2.55 (s, 6H), 2.24 (s, 3H), 1.06 (d, 6.8 Hz, 3H)

APCI-MS m/z: 364.1 [MH+].

### EXAMPLE 134

### N-[2-(3,5-Dimethoxyphenoxy)-1-methylethyl]-2,4,6-trimethylbenzenesulfonamide

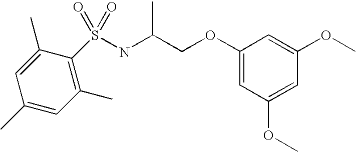

APCI-MS m/z: 394.1 [MH+].

LC (method A) rt=6.1 min. UV 254 nm.

### EXAMPLE 135

### N-[2-(4-Cyano-2-methoxyphenoxy)-1-methylethyl]-2,4,6-trimethylbenzenesulfonamide

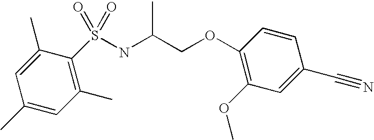

APCI-MS m/z: 389.1 [MH+].

LC (method A) rt=5.7 min. UV 254 nm.

### EXAMPLE 136

### N-{2-[(2-Bromopyridin-3-yl)oxy]-1-methylethyl}-2,4,6-trimethylbenzenesulfonamide

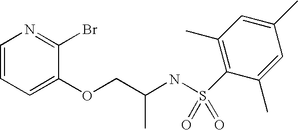

APCI-MS m/z: 413.1, 415.1 [MH+].

LC (method A) rt=5.5 min. UV 254 nm.

### EXAMPLE 137

### 2,4,6-Trimethyl-N-{1-methyl-2-[(2-methylpyridin-3-yl)oxy]ethyl}benzenesulfonamide

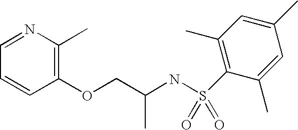

APCI-MS m/z: 349.2 [MH+].

LC (method A) rt=3.8 min. UV 254 nm.

### EXAMPLE 138

### 2-{2-[(Mesitylsulfonyl)amino]-propoxy}-N-methylbenzamide

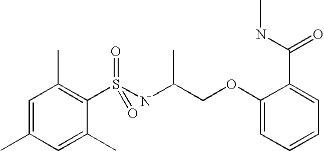

¹H NMR (399.988 MHz, CDCl3) δ 8.14 (dd, J=7.8, 1.7 Hz, 1H), 7.84 (s, 1H), 7.38 (dd, J=5.6, 1.8 Hz, 1H), 7.09 (t, J=7.5 Hz, 1H), 6.94 (s, 2H), 6.82 (d, J=8.4 Hz, 1H), 4.94-4.82 (m, 1H), 3.99-3.96 (m, 2H), 3.88-3.78 (m, 1H), 3.06 (d, J=4.9 Hz, 3H), 2.65 (s, 6H), 2.29 (s, 3H), 1.12 (d, J=6.8 Hz, 3H)

APCI-MS m/z: 391.2 [MH+].

### EXAMPLE 139

### 2-{2-[(Mesitylsulfonyl)amino]propoxy}benzamide

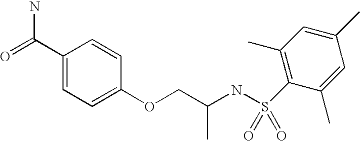

¹H NMR (299.944 MHz, CDCl₃) δ 7.73 (dd, 6.9, 1.9 Hz, 2H), 6.91 (s, 2H), 6.77 (d, J=.2 Hz, 2H), 5.03 (d, J=7.9 Hz, 1H), 3.89-3.74 (m, 2H), 3.75-3.63 (m, 1H), 6.16-5.63 (m, H), 2.65 (s, 6H), 2.27 (s, 3H), 1.26 (d, J=6.8 Hz, 3H)

APCI-MS m/z: 377.3 [MH+].

### EXAMPLE 140

### N-{2-[4-(1H-Imidazol-1-yl)phenoxy]-1-methylethyl}-2,4,6-trimethylbenzenesulfonamide

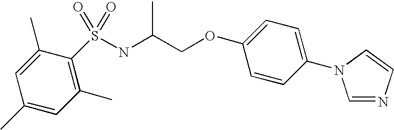

¹H NMR (299.944 MHz, CDCl₃) δ9.02 (s, 1H), 7.58 (s, 1H), 7.46-7.39 (m, 3H), 6.96 (d, J=.3 Hz, 4H), 5.10 (d, J=8.1 Hz, 1H), 3.92 (t, J=4.2 Hz, 2H), 3.77-3.62 (m, 1H), 2.67 (s, H), 2.29 (s, 3H), 1.26 (d, J=6.8 Hz, 3H)

APCI-MS m/z: 400.2 [MH+].

### EXAMPLE 141

### N-[(1S)-2-(3,4-Dimethoxyphenoxy)-1-methylethyl]-2,4,6-trimethylbenzenesulfonamide

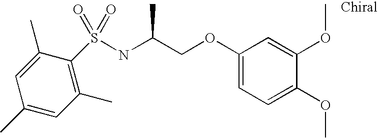

¹H NMR (299.946 MHz, DMSO) δ 7.67 (d, J=8.4 Hz, 1H), 7.01 (s, 2H), 6.77 (d, J=8.8 Hz, H), 6.29 (d, J=2.8 Hz, 1H), 6.20 (dd, J=8.6, 2.8 Hz, 1H), 3.75-3.55 (m, 9H), 2.55 (s, 6H), .24 (s, 3H), 1.06 (d, J=6.6 Hz, 3H)

APCI-MS m/z: 394.3 [MH+].

### EXAMPLE 142

### N-(2-{2-[(Mesitylsulfonyl)amino]propoxy}phenyl)acetamide

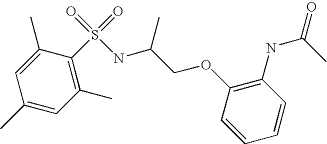

¹H NMR (299.944 MHz, CDCl₃) δ 8.58 (s, 1H), 8.41-8.36 (m, 1H), 6.99-6.93 (m, 4H),

6.75-6.69 (m, 1H), 4.88 (s, 1H), 3.96 (d, J=5.7 Hz, 1H), 3.74 (d, J=4.6 Hz, 2H), 2.66 (s, H), 2.31 (s, 3H), 2.25 (s, 3H), 1.09 (d, J=6.4 Hz, 3H)

APCI-MS m/z: 391.2 [MH+].

### EXAMPLE 143

### N-{2-[(6-Chloropyridin-3-yl)oxy]-1-methylethyl}-2,4,6-trimethylbenzenesulfonamide

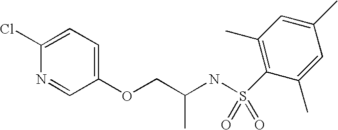

APCI-MS m/z: 369.2 [MH+].

LC (method A) rt=5.6 min. UV 254 nm.

### EXAMPLE 144

### N-[(1S)-2-(2H-Indazol-3-yloxy)-1-methylethyl]-2,4,6-trimethylbenzenesulfonamide

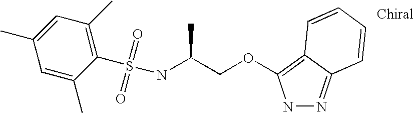

¹H NMR (399.99 MHz, DMSO) δ 11.79 (s, 1H), 7.72 (d, J=8.6 Hz, 1H), 7.36 (d, J=8.0 Hz, H), 7.30 (d, J=3.5 Hz, 2H), 6.98 (dt, 8.0, 3.9 Hz, 1H), 6.88 (s, 2H), 4.14-4.00 (m, 2H), .63 (quintet, J=6.9 Hz, 1H), 2.54 (s, 6H), 2.16 (s, 3H), 1.11 (d, J=6.7 Hz, 3H)

APCI-MS m/z: 374.1 [MH+].

### EXAMPLE 145

### -Methyl-N-[3-phenyl-1-(trifluoromethyl)propyl]benzenesulfonamide

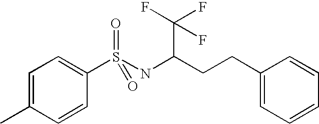

### -Methyl-N-[(1Z)-3-phenylpropylidene]benzenesulfonamide

A mixture of 4-methylbenzenesulfonamide (10 mmole, 1.71 g), 3-phenylpropanal 10 mmole, 1.34 g) and sodium p-toluenesulfinate (11 mmole, 1.78 g) in formic acid (15 mL) and water (15 mL) was stirred over night. The resulting white precipitate was filtered off, washed with water (2×10 mL), pentane (10 mL) and dissolved in dichloromethane (100 mL). Saturated NaHCO₃/aq (70 mL) was added and the mixture was stirred vigorously for 2 hours. The organic phase was decanted and the aqueous phase was extracted with CH₂Cl₂. The combined phases was dried and evaporated to dryness and used in the next step without any further purification.

### -Methyl-N-[3-phenyl-1-(trifluoromethyl)propyl]benzenesulfonamide

TBAT (1.1 mmole, 594 mg) was dissolved in dry THF (12 mL) and cooled to 0° C. under inert conditions. In a separate flask 4-methyl-N-[(1Z)-3-phenylpropylidene]-benzenesulfonamide (1 mmole, 287 mg) and trimethyl(trifluoromethyl)silane (1.2 mmole, 70 mg) were dissolved in dry THF (10 mL) and slowly added to the TBAT-solution. The mixture was stirred for 45 min at 0° C. before it was quenched with sat. NH₄Cl/aq (6 mL). At room temperature the mixture was extracted with ethylacetate. The organic phase was dried, concentrated and purified on a silica gel column chromatography (heptane-ethyl acetate).

¹H NMR (299.946 MHz, DMSO) δ 8.71 (d, J=8.6 Hz, 1H), 7.88 (dt, J=6.5, 1.9 Hz, 2H), .54 (d, J=7.9 Hz, 2H), 7.42-7.26 (m, 3H), 7.16-7.12 (m, 2H), 4.18-4.00 (m, 1H), 2.55-2.34 (m, 5H), 2.06-1.91 (m, 1H), 1.88-1.70 (m, 1H)

¹⁹F NMR (470.314 MHz, DMSO) δ −74.42 (d)

### EXAMPLE 146

### N-[(1S)-2-(Isoquinolin-5-yloxy)-1-methylethyl]-2,4-dimethylbenzenesulfonamide

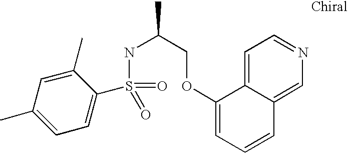

### 2,4-Dimethylbenzenesulfonyl chloride

2,4-Dimethylbenzenesulfonic acid (10 mmole, 1.86 g), DIEA (10 mmole, 1.7 mL) and cyanuric chloride (10 mmole, 1.84 g) were dissolved in acetone (40 mL) and the reaction mixture was refluxed overnight. After cooling to room temperature the mixture was filtered through a Celite pad. Solvent was removed by evaporation under reduced pressure. The product was used in the next step without any further purification.

### N-[(1S)-2-(Isoquinolin-5-yloxy)-1-methylethyl]-2,4-dimethylbenzenesulfonamide

The sulfonamide coupling was performed as described in Example 96 using the corresponding starting materials.

APCI-MS m/z: 371.2 [MH+].

LC (method A) rt=3.8 min. UV 254 nm.

Examples 147 to 153 were synthesised by a method analogous to that described in Example 146 using the corresponding starting materials.

### EXAMPLE 147

### N-[(1S)-2-(Isoquinolin-5-yloxy)-1-methylethyl]-3,4-dimethylbenzenesulfonamide

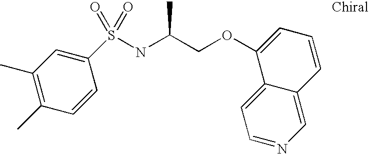

APCI-MS m/z: 371.2 [MH+].

LC (method A) rt=3.8 min. UV 254 nm.

### EXAMPLE 148

### N-[(1S)-2-(Isoquinolin-5-yloxy)-1-methylethyl]-2,5-dimethylbenzenesulfonamide

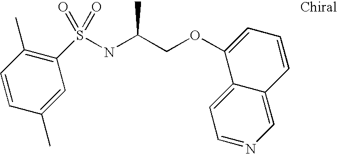

APCI-MS m/z: 371.2 [MH+].

LC (method A) rt=3.8 min. UV 254 nm.

### EXAMPLE 149

### 2,4-Dimethyl-N-[(1S)-1-methyl-2-(quinolin-5-yloxy)ethyl]benzenesulfonamide

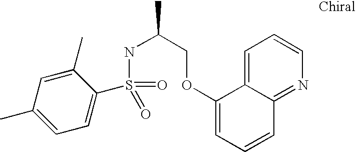

APCI-MS m/z: 371.2 [MH+].

LC (method A) rt=3.8 min. UV 254 nm.

### EXAMPLE 150

### 2,4-Dimethyl-N-[(1S)-1-methyl-2-(quinolin-5-yloxy)ethyl]benzenesulfonamide

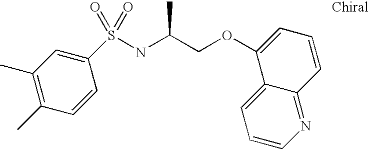

APCI-MS m/z: 371.2 [MH+].

LC (method A) rt=3.8 min. UV 254 nm.

### EXAMPLE 151

### -[((2S)-2-{[(2,4-Dimethylphenyl)sulfonyl]amino}propyl)oxy]benzamide

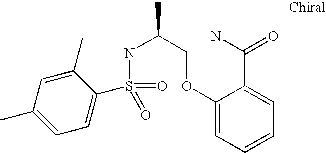

APCI-MS m/z: 363.2 [MH+].

LC (method A) rt=4.5 min. UV 254 nm.

### EXAMPLE 152

### ,5-Dimethyl-N-[(1)-1-methyl-2-quinolin-5-yloxy)ethyl]benzenesulfonamide

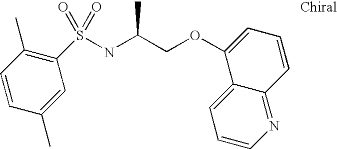

APCI-MS m/z: 371.2 [MH+].

LC (method A) rt=3.8 min. UV 254 nm.

### EXAMPLE 153

### -[((2S)-2-{[(3,4-Dimethylphenyl)sulfonyl]amino}propyl)oxy]benzamide

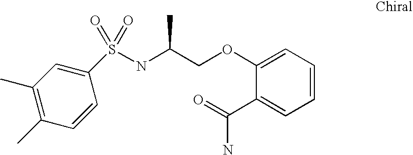

APCI-MS m/z: 363.2 [MH+].

LC (method A) rt=4.5 min. UV 254 nm.

Examples 154 to 158 were synthesised by a method analogous to that described in Example 96, “Sulfonamide coupling”, using the corresponding starting materials.

### EXAMPLE 154

### -(2-Anilinoethyl)-2,4,6-trimethylbenzenesulfonamide

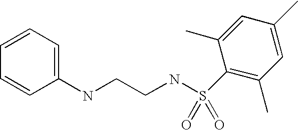

APCI-MS m/z: 319.4 [MH+].

LC (method A) rt=4.6 min. UV 254 nm.

### EXAMPLE 155

### -[2-(2,6-Dimethylphenoxy)-1-methylethyl]-4-(trifluoromethyl)benzenesulfonamide

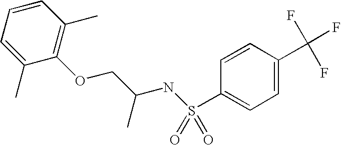

LC (method A) rt=5.4 min. UV 254 nm.

### EXAMPLE 156

### N-(2-Anilinoethyl)-4′-fluorobiphenyl-4-sulfonamide

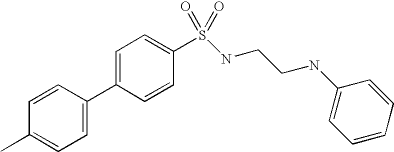

APCI-MS m/z: 371.0 [MH+].

LC (method A) rt=5.0 min. UV 254 nm.

### EXAMPLE 157

### N-(2-Anilinoethyl)-4-methoxy-2,3,6-trimethylbenzenesulfonamide

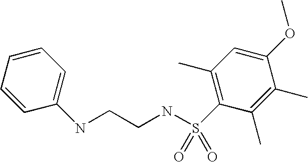

APCI-MS m/z: 349.1 [MH+].

LC (method A) rt=4.7 min. UV 254 nm.

### EXAMPLE 158

### N-(2-Anilinoethyl)-4-bromo-2-methylbenzenesulfonamid

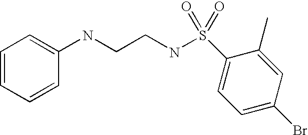

APCI-MS m/z: 369.1, 371.1 [MH+].

LC (method A) rt=4.8 min. UV 254 nm.

### EXAMPLE 159

### 1-(4-Fluorophenyl)-N-[(1S)-2-(isoquinolin-5-yloxy)-1-methylethyl]-3,5-dimethyl-1H-pyrazole-4-sulfonamide

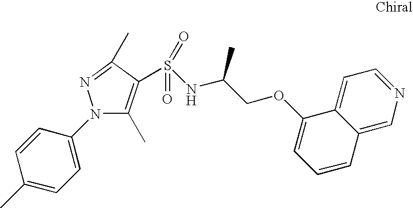

### 1-(4-Fluorophenyl)-3,5-dimethyl-1H-pyrazole

4-Fluorophenylhydrazine hydrochloride (3 mmole, 488 mg) and acetylacetone 3 mmole, 310 μL) were refluxed in ethanol (25 mL) for 1 hour before the reaction mixture was evaporated to dryness. The residue was used in the next step without any purification.

### 1-(4-Fluorophenyl)-3,5-dimethyl-1H-pyrazole-4-sulfonyl chloride

1-(4-Fluorophenyl)-3,5-dimethyl-1H-pyrazole (app. 3 mmole) was dissolved in chloroform (40 mL). Chlorosulfonic acid (30 mmole, 2 mL) was added dropwise and the reaction mixture was refluxed for 2 hours. After cooling the mixture to room temperature sulfuryl chloride (25 mmole, 2 mL) was added. The reaction mixture was refluxed for 3 hours before it was diluted with chloroform and washed with water. The organic phase was dried, concentrated and purified on a silica gel column chromatography (heptane-ethyl acetate).

APCI-MS m/z: 288.9 [MH+].

### 1-(4-Fluorophenyl)-N-[(1S)-2-(isoquinolin-5-yloxy)-1-methylethyl]-3,5-dimethyl-1H-pyrazole-4-sulfonamide

Amine preparation and Sulfonamide coupling were conducted using a method analogous to that described in Example 96.

¹H NMR (399.99 MHz, DMSO) δ 9.53 (s, 1H), 8.55 (d, J=6.1 Hz, 1H), 8.31 (d, J=6.1 Hz, H), 7.99 (d, J=8.1 Hz, 1H), 7.84 (d, J=8.3 Hz, 1H), 7.72 (t, J=8.0 Hz, 1H), 7.36 (mult, H), 4.12-4.01 (m, 2H), 3.75-3.69 (m, 1H), 2.37 (s, 3H), 2.32 (s, 3H), 1.24 (t, J=6.8 Hz, H)

APCI-MS m/z: 455.1 [MH+].

### EXAMPLE 160

### N-[(1S)-2-(Isoquinolin-5-yloxy)-1-methylethyl]-3,5-dimethyl-1-phenyl-1-pyrazole-4-sulfonamide

Example 160 was synthesised using a method analogous to Example 159.

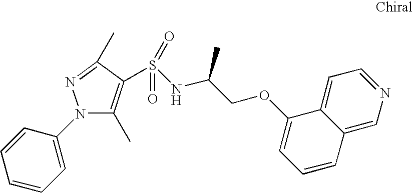

¹H NMR (399.99 MHz, DMSO) δ δ9.50 (s, 1H), 8.53 (d, J=6.1 Hz, 1H), 8.28 (d, J=6.1 Hz, H), 7.98 (d, J=8.2 Hz, 1H), 7.82 (d, J=8.2 Hz, 1H), 7.71 (t, J=8.0 Hz, 1H), 7.54-7.43 (m, 3H), 7.32 (dd, J=6.4, 1.8 Hz, 3H), 4.06 (quintet, J=4.7 Hz, 2H), 3.75 (q, J=6.4 Hz, H), 2.39 (s, 3H), 2.34 (s, 3H), 1.25 (d, J=6.8 Hz, 3H)

APCI-MS m/z: 437.1 [MH+].

### EXAMPLE 161

### N,2,4,6-Tetramethyl-N-[(1S)-1-methyl-3-phenylpropyl]benzenesulfonamide

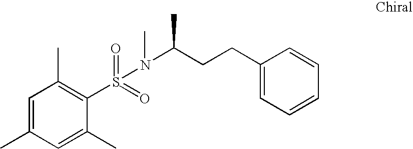

2,4,6-Trimethyl-N-[(1S)-1-methyl-3-phenylpropyl]benzenesulfonamide (109 mg, .33 mmol) and potassium carbonate (272 mg, 2.0 mmol) was dissolved in DMF (1 ml), the solution was cooled to 0° C. and iodomethane (41 μl, 0.66 mmol) was added dropwise. The reaction mixture was stirred for 15 h at ambient temperature, dispersed between dichloromethane and water and extracted with dichloromethane. The combined organic phases were dried over sodium sulphate, filtered and evaporated.

¹H NMR (299.944 MHz, CDCl₃) δ 7.26-7.15 (m, 3H), 7.08-7.04 (m, 2H), 6.93 (s, 2H), .75 (q, 1H), 2.74 (s, 3H), 2.58 (s, 6H), 2.56-2.40 (m, 2H), 2.31 (s, 3H), 1.86-1.64 (m, 2H), .19 (d, 3H).

GC-MS m/z: 345 [M].

LC (method B) rt=16.2 min. UV 254 nm.

### EXAMPLE 162

### 2,4,6-Trimethyl-N-{1-[(quinolin-5-yloxy)methyl]propyl}benzenesulfonamide

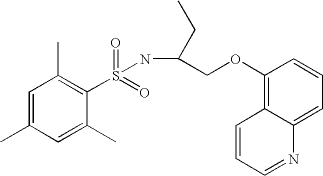

The title compound was obtained from 2-mesitylenesulfonyl chloride, 2-aminobutan-ol and quinolin-5-ol by a method analogous to that described in Example 77.

¹H NMR (400 MHz, CDCl₃) δ 8.96 (dd, 1H), 8.52 (d, 1H), 7.74 (d, 1H), 7.53 (s, 1H), 7.39 (m, 1H), 6.83 (s, 2H), 6.68 (d, 1H), 5.50 (bs, 1H), 4.12 (dd, 1H), 3.98 (dd, 1H), 3.63 (m, 1H), 2.63 (s, 6H), 2.24 (s, 3H), 1.75 (m, 2H), 0.91 (t, 3H).

APCI-MS m/z: 399 [MH+].

LC (method B) rt=8.1 min. UV 254 nm.

### EXAMPLE 163

### -Chloro-2-{2-[(mesitylsulfonyl)amino]butoxy}benzamide

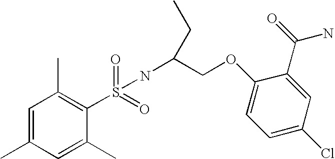

The title compound was obtained from 2-mesitylenesulfonyl chloride, 2-aminobutan-ol and 5-chloro-2-hydroxybenzamide by a method analogous to that described in Example 7.

¹H NMR (400 MHz, dimethylsulfoxide-d₆) δ 7.73 (d, 1H), 7.43 (dd, 1H), 6.97 (d, 1H), 6.93 (s, 2H), 3.95 (m, 2H), 3.36 (m, 1H), 2.53 (s, 6H), 2.21 (s, 3H), 1.54-1.35 (m, 2H), 0.68 (t, H).

APCI-MS m/z: 425/427 (3:1) [MH+].

LC (method B) rt=11.7 min. UV 254 nm.

Examples 164-184 were synthesised by a method analogous to that described in Example 17 using the corresponding starting materials.

### EXAMPLE 164

### 2,4-Dichloro-6-methyl-N-[(1S)-1-methyl-2-(quinolin-5-yloxy)ethyl]benzenesulfonamide

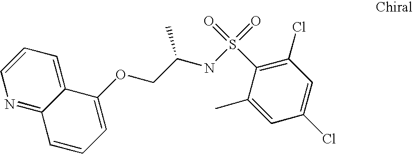

APCI-MS m/z: 425/427 [MH+].

LC (method A) rt=4.0 min. UV 254 nm.

### EXAMPLE 165

### 5-Chloro-2-{[(2S)-2-({[4-(4-fluorophenoxy)phenyl]sulfonyl}amino)propyl]oxy}benzamide

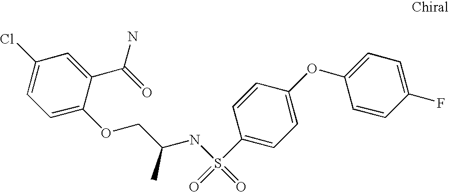

APCI-MS m/z: 479/481 (3:1) [MH+].

LC (method A) rt=5.6 min. UV 254 nm

### EXAMPLE 166

### 5-Chloro-2-{[(2S)-2-({[4-(4-methoxyphenoxy)phenyl]sulfonyl}amino)propyl]oxy}-benzamide

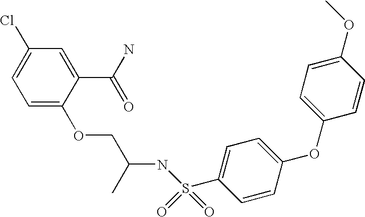

APCI-MS m/z: 491/493 (3:1) [MH+].

LC (method A) rt=5.5 min. UV 254 nm

### EXAMPLE 167

### 5-Chloro-2-{[(2S)-2-({[3-(4-chlorophenoxy)phenyl]sulfonyl}amino)propyl]oxy}benzamide

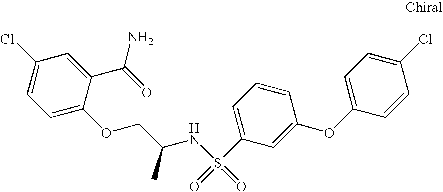

APCI-MS m/z: 495/497 [MH+].

LC (method A) rt=5.9 min. UV 254 nm

### EXAMPLE 168

### 2,4,5-Trichloro-N-[(1S)-1-methyl-2-(quinolin-5-yloxy)ethyl]benzenesulfonamide

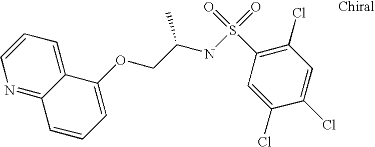

APCI-MS m/z: 445/447 [MH+].

LC (method A) rt=4.2 min. UV 254 nm

### EXAMPLE 169

### Chloro-2-{[(2S)-2-({[3-(3,4-dichlorophenoxy)phenyl]sulfonyl}amino)propyl]oxy}-benzamide

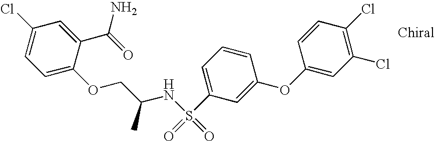

APCI-MS m/z: 529/531 [MH+].

LC (method A) rt=6.2 min. UV 254 nm

### EXAMPLE 170

### -(4-Chlorophenoxy)-N-[(1S)-1-methyl-2-(quinolin-5-yloxy)ethyl]benzenesulfonamide

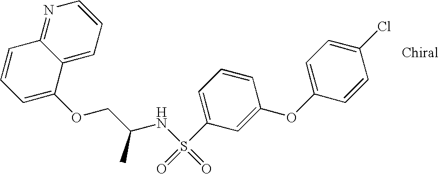

APCI-MS m/z: 469/471 (3:1) [MH+].

LC (method A) rt=4.9 min. UV 254 nm

### EXAMPLE 171

### -Chloro-2-[((2S)-2-{[(2,4-dichloro-5-fluorophenyl)sulfonyl]-amino}propyl)oxy]benzamide

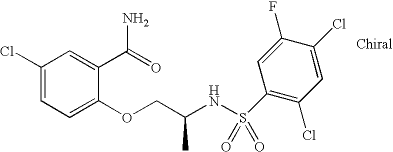

APCI-MS m/z: 455/457 [MH+].

LC (method A) rt=5.1 min. UV 254 nm

### EXAMPLE 172

### 5-Chloro-2-{[(2S)-2-({[3-(4-methoxyphenoxy)phenyl]sulfonyl}amino)propyl]oxy}benzamide

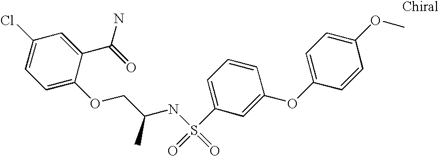

APCI-MS m/z: 491/493 (3:1) [MH+].

LC (method A) rt=5.5 min. UV 254 nm

### EXAMPLE 173

### 5-Chloro-2-[((2S)-2-{[(2-methoxy-4-methylphenyl)sulfonyl]amino}propyl)oxy]benzamide

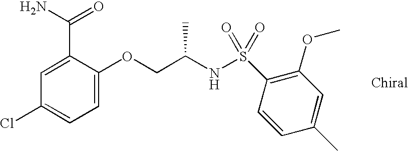

APCI-MS m/z: 413/415 (3:1) [MH+].

LC (method A) rt=4.8 min. UV 254 nm

### EXAMPLE 174

### 4-(4-Fluorophenoxy)-N-[(1S)-1-methyl-2-(quinolin-5-yloxy)ethyl]benzenesulfonamide

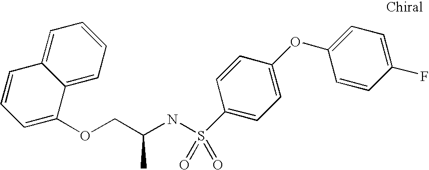

APCI-MS m/z: 453 [MH+].

LC (method A) rt=4.6 min. UV 254 nm

### EXAMPLE 175

### 5-Chloro-2-[((2S)-2-{[(5-chloro-2-methoxyphenyl)sulfonyl]amino}propyl)oxy]benzamide

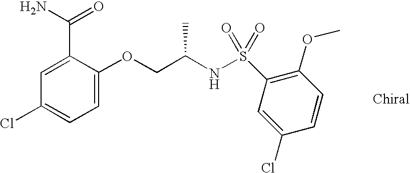

APCI-MS m/z: 433/435 (3:1) [MH+].

LC (method A) rt=5.0 min. UV 254 nm

### EXAMPLE 176

### -Cyano-N-[(1S)-1-methyl-2-(quinolin-5-yloxy)ethyl]benzenesulfonamide

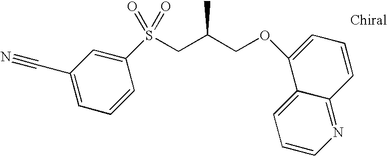

APCI-MS m/z: 368 [MH+].

LC (method A) rt=3.2 min. UV 254 nm

### EXAMPLE 177

### 2,4-Dichloro-5-fluoro-N-[(1S)-1-methyl-2-(quinolin-5-yloxy)ethyl]benzenesulfonamide

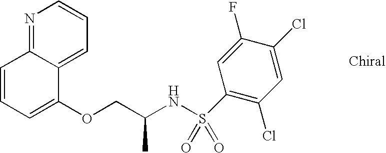

APCI-MS m/z: 429/431 [MH+].

LC (method A) rt=4.0 min. UV 254 nm

### EXAMPLE 178

### 2-[((2S)-2-{[(5-Bromo-2-methoxyphenyl)sulfonyl]amino}propyl)oxy]-5-chlorobenzamide

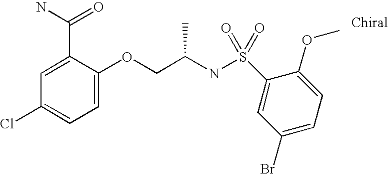

APCI-MS m/z: 477/479 (1:1) [MH+].

LC (method A) rt=5.0 min. UV 254 nm

### EXAMPLE 179

### 5-Chloro-2-[((2S)-2-{[(2-methoxy-5-methylphenyl)sulfonyl]amino}propyl)oxy]benzamide

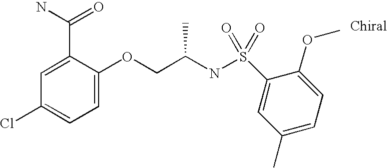

APCI-MS m/z: 413/415 (3:1) [MH+].

LC (method A) rt=4.8 min. UV 254 nm

### EXAMPLE 180

### 5-Chloro-2-{[(2S)-2-({[4′-trifluoromethyl)biphenyl-4-yl]sulfonyl}amino)propyl]oxy}-benzamide

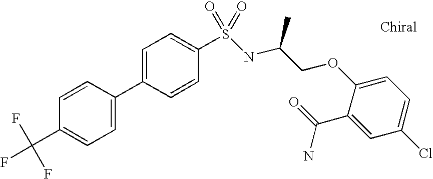

APCI-MS m/z: 513/515 (3:1) [MH+].

LC (method A) rt=6.0 min. UV 254 nm

### EXAMPLE 181

### -(4-Methoxyphenoxy)-N-[(1S)-1-methyl-2-(quinolin-5-yloxy)ethyl]benzenesulfonamide

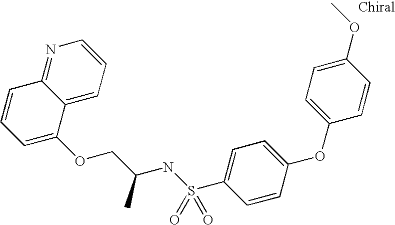

APCI-MS m/z: 465 [MH+].

LC (method A) rt=4.5 min. UV 254 nm

### EXAMPLE 182

### 5-Chloro-2-[((2S)-2-{[(6-phenoxypyridin-3-yl)sulfonyl]amino}propyl)oxy]benzamide

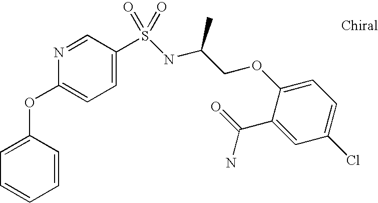

APCI-MS m/z: 462/464 (3:1) [MH+].

LC (method A) rt=5.1 min. UV 254 nm

### EXAMPLE 183

### 5-Bromo-6-chloro-N-[(1S)-1-methyl-2-(quinolin-5-yloxy)ethyl]pyridine-3-sulfonamide

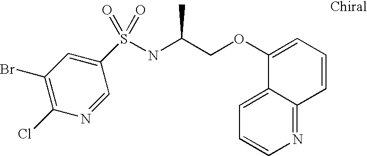

APCI-MS m/z: 456/458 [MH+].

LC (method A) rt=3.7 min. UV 254 nm

### EXAMPLE 184

### 5-Bromo-2-methoxy-N-[(1S)-1-methyl-2-(quinolin-5-yloxy)ethyl]benzenesulfonamide

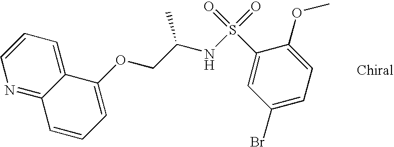

APCI-MS m/z: 451/453 (1:1) [MH+].

LC (method A) rt=4.0 min. UV 254 nm

### EXAMPLE 185

### N-[(1S)-1-Methyl-2-(quinolin-5-yloxy)ethyl]-1-benzothiophene-2-sulfonamide

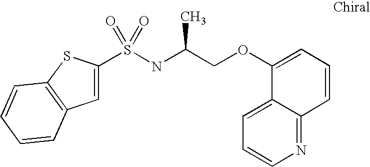

To a solution of (2S)-1-(quinolin-5-yloxy)propan-2-amine in DMF (100 μL 0.3M/DMF) was added diisopropylethylamine (120 μL 0.3M/THF) followed by 1-benzothiophene-2-sulfonyl chloride (120 μL 0.3M/THF). The reaction mixture was stirred overnight at ambient temperature, evaporated to dryness under reduced pressure and purified on HPLC-C₁₈.

APCI-MS m/z: 399 [MH+].

LC (method A) rt=3.9 min. UV 254 nm

Examples 186-194 were synthesised by a method analogous to that described in Example 185 using the corresponding starting materials.

### EXAMPLE 186

### 5-Chloro-2-[((2S)-2-{[(2,4-dimethoxyphenyl)sulfonyl]amino}propyl)oxy]benzamide

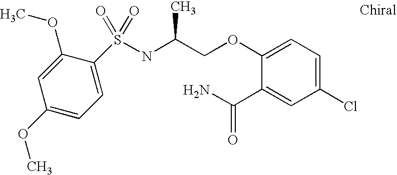

APCI-MS m/z: 429/431 (3:1) [MH+].

LC (method A) rt=4.6 min. UV 254 nm

### EXAMPLE 187

### -({(2S)-2-[(1-Benzothien-2-ylsulfonyl)amino]propyl}oxy)-5-chlorobenzamide

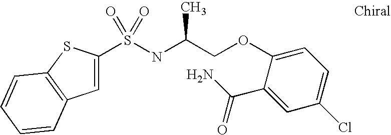

APCI-MS m/z: 425/427 (3:1) [MH+].

LC (method A) rt=5.1 min. UV 254 nm

### EXAMPLE 188

### 5-Chloro-2-[((2S)-2-{[(4-methoxy-2,3,6-trimethylphenyl)sulfonyl]amino}propyl)oxy]-benzamide

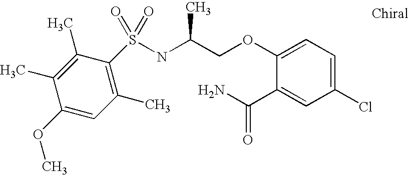

APCI-MS m/z: 441/443 (3:1) [MH+].

LC (method A) rt=5.2 min. UV 254 nm

### EXAMPLE 189

### 5-Chloro-2-[((2S)-2-{[(5-fluoro-3-methyl-1-benzothien-2-yl)sulfonyl]amino}propyl)oxy]-benzamide

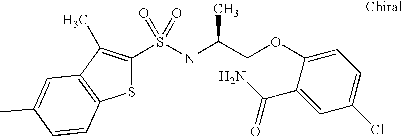

APCI-MS m/z: 457/459 (3:1) [MH+].

LC (method A) rt=5.3 min. UV 254 nm

### EXAMPLE 190

### 5-Chloro-2-[((2S)-2-{[(5-chloro-3-methyl-1-benzothien-2-yl)sulfonyl]amino}propyl)oxy]-benzamide

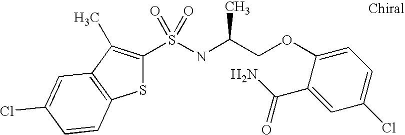

APCI-MS m/z: 473/475 [MH+].

LC (method A) rt=4.0 min. UV 254 nm

### EXAMPLE 191

### -{[(2S)-2-({[4-Bromo-2-(trifluoromethoxy)phenyl]sulfonyl}amino)propyl]oxy}-5-chlorobenzamide

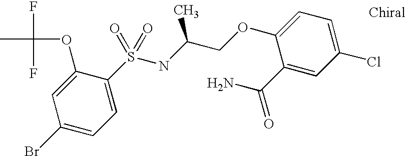

APCI-MS m/z: 531/532 [MH+].

LC (method A) rt=5.5 min. UV 254 nm

### EXAMPLE 192

### 2,4,6-Trichloro-N-[(1S)-1-methyl-2-(quinolin-5-yloxy)ethyl]benzenesulfonamide

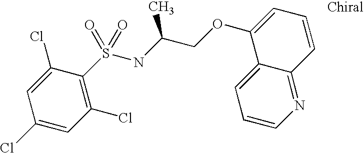

APCI-MS m/z: 445/447 [MH+].

LC (method A) rt=4.0 min. UV 254 nm

### EXAMPLE 193

### -Methoxy-2,3,6-trimethyl-N-[(1S)-1-methyl-2-(quinolin-5-yloxy)ethyl]-benzenesulfonamide

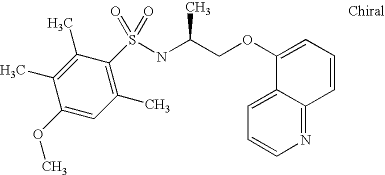

APCI-MS m/z: 415 [MH+].

LC (method A) rt=4.0 min. UV 254 nm

### EXAMPLE 194

### -Bromo-N-[(1)-1-methyl-2-(quinolin-5-yloxy)ethyl]-2-(trifluoromethoxy)-benzenesulfonamide

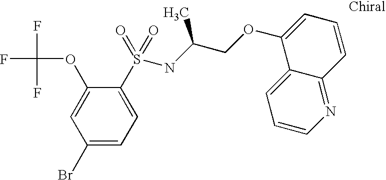

APCI-MS m/z: 505/507 (1:1) [MH+].

LC (method A) rt=4.2 min. UV 254 nm

### EXAMPLE 195

### Human Glucocorticoid Receptor (GR) Assay

The assay is based on a commercial kit from Panvera/Invitrogen (Part number P2893). The assay technology is fluorescence polarization. The kit utilises recombinant human GR (Panvera, Part number P2812), a Fluoromone™ labelled tracer (GS Red, Panvera, Part number P2894) and a Stabilizing Peptide 10X (Panvera, Part number P2815). The GR and Stabilizing Peptide reagents are stored at −70° C. while the GS Red is stored at −20° C. Also included in the kit are 1M DTT (Panvera, Part number P2325, stored at −20° C.) and GR Screening buffer 10X (Panvera, Part number P2814, stored at −70° C. initially but once thawed stored at room temperature). Avoid repeated freeze/thaws for all reagents. The GR Screening buffer 10X comprises 100 mM potassium phosphate, 200 mM sodium molybdate, 1 mM EDTA and 20% DMSO.

Test compounds (1 μL) and controls (1 μL) in 100% DMSO were added to black polystyrene 384-well plates (Greiner low volume black flat-bottom, part number 784076). % control was 100% DMSO and 100% control was 10 μM Dexamethasone. Background solution (8 μL; assay buffer 10X, Stabilizing Peptide, DTT and ice cold MQ water) was added to the background wells. GS Red solution (7 μL; assay buffer 10X, Stabilizing Peptide, DTT, GS Red and ice cold water) was added to all wells except background wells. GR solution (7 μL; assay buffer 10X, Stabilizing Peptide, DTT, GR and ice cold water) was added to all wells. The plate was sealed and incubated in a dark at room temperature for 2 hours. The plate was read in an Analyst plate reader (LJL Biosystems/Molecular Devices Corporation) or other similar plate reader capable of recording fluorescence polarization (excitation wavelength 530 nm, emission wavelength 590 nM and a dichroic mirror at 561 nm). The IC50 values were calculated using XLfit model 205.

| | GRhuFL_FP_v2 (GR-binders) |
|---|---|
| Example No | IC50 (μM) |
| 1 | 1.4 |
| 2 | 1.9 |
| 3 | 0.40 |
| 4 | 0.064 |
| 5 | 0.64 |
| 6 | 0.7 |
| 7 | 0.70 |
| 8 | 1.2 |
| 9 | 1.6 |
| 10 | 0.60 |
| 11 | 2.2 |
| 12 | 6.0 |
| 13 | 2.2 |
| 14 | 1.7 |
| 15 | 6.3 |
| 16 | 4.4 |
| 19 | 0.54 |
| 32 | 0.090 |
| 34 | 3.0 |
| 77 | 0.017 |
| 78 | 0.023 |
| 79 | 0.14 |
| 80 | 0.23 |
| 81 | 0.37 |
| 82 | 3.4 |
| 83 | 8.9 |
| 123 | 0.018 |
| 124 | 0.020 |
| 125 | 0.042 |
| 126 | 0.075 |
| 160 | 0.096 |

1. A compound of formula (I): 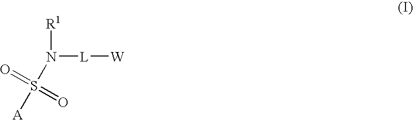
wherein:
A is phenyl, naphthyl pyridinyl, furyl, thienyl, isoxazolyl, pyrazolyl, benzthienyl, quinolinyl or isoquinolinyl, and A is optionally substituted by halo, C₁₋₆ alkyl, C₁₋₆ alkoxy, C₁₋₄ alkylthio, C₁₋₄ fluoroalkyl, C₁₋₄ fluoroalkoxy, pyridinyloxy, benzyloxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), C(O)NH₂, C(O)NH(C₁₋₄ alkyl), C(O)N(C₁₋₄ alkyl)₂, NHC(O)(C₁₋₄ alkyl), NR¹⁰R¹¹, phenoxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁴R¹⁵), phenyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁶R¹⁷), pyridinyloxy (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O)N(C₁₋₄ alkyl)₂, NHC(O)(C₁₋₄ alkyl) or NR¹⁸R¹⁹) or pyrazolyl (optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, C(O)(C₁₋₄ alkyl), benzyloxy, C(O)NH₂, C(O)NH(C₁₋₄ alkyl), C(O))N(C₁₋₄ alkyl)₂, NHC(O)(C₁₋₄ alkyl) or NR²⁰R²¹);
R¹⁰, R¹¹, R¹⁴, R¹⁵, R¹⁶, R¹⁷, R¹⁸, R¹⁹, R²⁰ and R²¹ are, independently, hydrogen, C₁₋₄ alkyl or C₃₋₇ cycloalkyl;
R¹ is hydrogen, C₁₋₆ alkyl, phenyl, pyridinylC(O), C₃₋₆ cycloalkyl, (C₃₋₆ cycloalkyl)CH₂ or C₃₋₄ alkenyl;
L is a bond, C₁₋₄ alkylene (optionally substituted by C₁₋₄ alkyl or C₁₋₄ haloalkyl), C₁₋₄ alkylene-NH (optionally substituted by C₁₋₄ alkyl or C₁₋₄ haloalkyl), CH₂C(O)NH, CH(CH₃)C(O)NH, C₁₋₄ alkylene-O (optionally substituted by C₁₋₄ alkyl or C₁₋₄ haloalkyl), C₁₋₄ alkylene-S (optionally substituted by C₁₋₄ alkyl or C₁₋₄ haloalkyl), C₁₋₄ alkylene-S(O) (optionally substituted by C₁₋₄ alkyl or C₁₋₄ haloalkyl) or C₁₋₄ alkylene-S(O)₂ (optionally substituted by C₁₋₄ alkyl or C₁₋₄ haloalkyl);
W is cyclohexyl, phenyl, methylenedioxyphenyl, thienyl, pyrazolyl, thiazolyl, isoxazolyl, pyridinyl, pyrimidinyl, pyridazinyl, pyrazinyl, 1,3,5-triazinyl, 1,2,3-triazinyl, 1,2,4-triazinyl, benzofuranyl, benzthienyl, indolyl, indolinyl, dihydroindolinyl, indazolyl, benzimidazolyl, benzoxazolyl, benzthiazolyl, quinolinyl, tetrahydroquinolinyl, isoquinolinyl, quinoxalinyl, quinazolinyl, cinnolinyl, phthalazinyl, [1,8]-naphthiridinyl, [1,6]naphthiridinyl, quinolin-2(1H)-onyl, isoquinolin-1(2H)-onyl, phthalazin-1(2H)-onyl, 1H-indazolyl, 1,3-dihydro-2H-indol-2-onyl, isoindolin-1-onyl, 3,4-dihydro-1H-isochromen-1-onyl or 1H-isochromen-1-onyl;
W is optionally substituted by halo, C₁₋₆ alkyl, C₁₋₆ alkoxy, C₁₋₄ alkylthio, C₁₋₄ fluoroalkyl, C₁₋₄ fluoroalkoxy, nitro, cyano, OH, C(O)₂H, C(O)₂(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, benzyloxy, imidazolyl C(O)(C₁₋₄ alkyl), C(O)NH₂, C(O)NH(C₁₋₄ alkyl), C(O)N(C₁₋₄ alkyl)₂, NHC(O)(C₁₋₄ alkyl) or NR¹²R¹³;
R¹² and R¹³ are, independently, hydrogen, C₁₋₄ alkyl or C₃₋₇ cycloalkyl;
or a pharmaceutically acceptable salt thereof.

2. A compound of formula (I) as claimed in claim 1 wherein A is phenyl (optionally substituted by halogen, C₁₋₄ alkyl, C₁₋₄ haloalkyl, C₁₋₄ alkoxy or C₁₋₄ haloalkoxy), pyridyl (optionally substituted by halogen, C₁₋₄ alkyl, C₁₋₄ haloalkyl, C₁₋₄ alkoxy or C₁₋₄ haloalkoxy) or pyrazolyl (optionally substituted by C₁₋₄ alkyl, C₁₋₄ haloalkyl or phenyl (itself optionally substituted by halogen, C₁₋₄ alkyl, C₁₋₄ haloalkyl, C₁₋₄ alkoxy or C₁₋₄ haloalkoxy)).

3. A compound of formula (I) as claimed in claim 1 wherein W is phenyl, pyridyl, indolyl, indazolyl, quinolinyl or isoquinolinyl.

4. A compound of formula (I) as claimed in claim 1, wherein W is optionally substituted by halogen, C₁₋₄ alkyl, CF₃, C₁₋₄ alkoxy, OCF₃, phenyl (itself optionally substituted by halogen, C₁₋₄ alkyl, CF₃, C₁₋₄ alkoxy or OCF₃) or C(O)NH₂.

5. A compound of formula (I) as claimed in claim 1 wherein L is C₃ alkylene (substituted by C₁₋₄ alkyl or C₁₋₄ haloalkyl), C₂₋₄ alkylene-NH (substituted by C₁₋₄ alkyl or C₁₋₄ haloalkyl), CH₂C(O)NH, CH(CH₃)C(O)NH, C₂₋₄ alkylene-O (substituted by C₁₋₄ alkyl or C₁₋₄ haloalkyl), C₂₋₄ alkylene-S (substituted by C₁₋₄ alkyl or C₁₋₄ haloalkyl), C₂₋₄ alkylene-S(O) (optionally substituted by C₁₋₄ alkyl or C₁₋₄ haloalkyl) or C₂₋₄ alkylene-S(O)₂ (optionally substituted by C₁₋₄ alkyl or C₁₋₄ haloalkyl).

6. A compound of formula (I) as claimed in claim 5 wherein L is CH(CH₃)CH₂CH₂, CH(CH₃)CH₂NH, CH(CH₃)CH₂O, CH(C₂H₅)CH₂CH₂, CH(C₂H₅)CH₂NH, CH(C₂H₅)CH₂O or CH(CF₃)CH₂CH₂.

7. A process for the preparation of a compound of formula (I) comprising coupling a compound of formula (II): 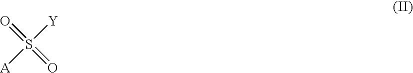
wherein Y is a leaving group, with a compound of Formula (III): 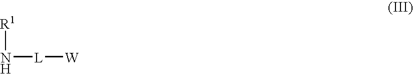
in a suitable solvent at a temperature in the range −10° C. to 50° C.

8. A pharmaceutical composition comprising a compound or formula (I) as claimed in claim 1, or a pharmaceutically acceptable salt thereof, and a pharmaceutically acceptable adjuvant, diluent or carrier.

9-10. (canceled)

11. A method of treating a glucocorticoid receptor mediated disease state in a mammal, which comprises administering to a mammal in need, of such treatment an effective amount of a compound of formula (I), or a pharmaceutically acceptable salt thereof.
